(19) United States
(12) Patent Application Publication
(10) Pub. No.: US 2025/0051733 A1
(43) Pub. Date: Feb. 13, 2025

(54) Method of Generating Phage Clones with Expanded Activity

(71) Applicant: The Government of the United States, as Represented by the Director of the Defense Health Agency, Fort Detrick, MD (US)

(72) Inventors: Mikeljon P. NIKOLICH, Tacoma Park, MD (US); Kevin BURKE, Silver Spring, MD (US); Andrey A. FILIPPOV, Gaithersburg, MD (US)

(21) Appl. No.: 18/744,397

(22) Filed: Jun. 14, 2024

Related U.S. Application Data
(60) Provisional application No. 63/531,942, filed on Aug. 10, 2023.

(51) Int. Cl. C12N 7/00; A01N 63/40; A01P 1/00; A61K 35/76; A61P 31/04

(52) U.S. Cl. CPC C12N 7/00; A01N 63/40; A01P 1/00; A61K 35/76; A61P 31/04; C12N 2795/10031; C12N 2795/10032; C12N 2795/10051

(57) ABSTRACT

The utility of directed-in vitro evolution of phages in a phage cocktail, i.e., phage training, using panels of multidrug resistant strains of P. aeruginosa or K. pneumoniae was demonstrated effective for the development of phages having broader host ranges of MDR P. aeruginosa or K. pneumoniae. The phage clones obtained by this method have lytic activity against more host strains than their parental phages. Sequencing results of the trained phages showed significant genetic changes from the parental phages. Some trained phages having lytic activity in an expanded range of MDR bacterial strains was proved to be stable, indicating the genetic changes that accumulated were not readily reversible. One of the phage clones showing host range stability was selected and incorporated into a previously used phage cocktail. The new phage cocktail provided improved therapeutic efficacy in a mouse model of wound infection. Taken together, these results show the utility of in vitro phage training in the development of more efficacious phage therapeutics to target the critical drug-resistant pathogens P. aeruginosa as well as K. pneumoniae. Based on these results, a new method for phage training and pharmaceutical composition comprising one of the trained phages is suggested.

### GOVERNMENT FUNDING SUPPORT

This invention was made with government support under grant no. W0350_20_WR, awarded by the Military Infectious Diseases Research Program and previous awards from the same program. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 14, 2024, is named “15969044US0_seq_listing.xml” and is 3,481,280 bytes in size. The sequence listing contained in this .XML file is part of the specification and is hereby incorporated by reference herein in its entirety.

### BACKGROUND

### 1. Field of the Invention

The invention relates to the general field of phage treatment for multidrug resistant (MDR) bacteria, and in particular to a method to generate phages with expanded activity and a pharmaceutical composition comprising at least one thereof for therapeutic treatment of MDR Pseudomonas aeruginosa and Klebsiella pneumoniae.

### 2. Background of the Invention

As antibiotic resistance continues to spread, multidrug-resistant (MDR), extensively drug-resistant (XDR) and pandrug-resistant (PDR) bacterial infections present a growing and serious global concern. Multidrug-resistant bacteria pathogens, especially the ESKAPEE group (gram-positive Enterococcus faecium and Staphylococcus aureus, and gram-negative Klebsiella pneumoniae, Acinetobacter baumannii, Pseudomonas aeruginosa, Enterobacter spp. and Escherichia coli), are responsible for over 10,000,000 infections a year worldwide and roughly 2.5 million deaths because of their high virulence and antibiotic resistance, and thus clinicians will be increasingly challenged to provide effective antibacterial treatments to patients suffering from.

MDR gram-negative bacteria present acute threats as they are spreading throughout the world and cause especially difficult-to-treat infections, with antibiotics losing efficacy.

Specifically, these pathogens are a significant challenge for wounded warfighters because they are found in mono- or polymicrobial combat wound infections, and the current antibiotic pipeline lacks new drugs with activity against these bacterial species. K. pneumoniae and P. aeruginosa are among the most prevalent gram-negative pathogens isolated from injured soldiers during Operation Iraqi Freedom and Operation Enduring Freedom, highlighting the need for novel therapeutics to serve the warfighter.

Considering the difficulty in finding a new compound showing antibiotic effects against a specific bacterial strain as well as drug developing cost and clinical safety concern thereof, phage therapy presents a powerful tool to supplement or possibly replace failing antibiotics for the treatment of these MDR infections since phages offer attractive characteristics for therapeutics, including replication at the site of infection, often capable of targeting difficult-to-reach places like biofilms, and minimal off-target killing because of the high strain specificity of most phages.

Phage therapy has great potential as an effective approach for treating drug-resistant bacteria. Bacteria and their phages have been evolving together for billions of years, and although bacteria can become resistant to phages, the phages can also evolve to overcome the resistance and infect the bacteria, which gives advantages over antibiotics. For successful phage therapy, it is necessary to continually isolate new phages that can overcome phage resistance of refractory bacterial strains. Although phages with such capability can be isolated from the environment, e.g., sea- and fresh water, sewage, or patient samples, it is not always possible or efficient to isolate phages with the clinical relevance.

There is another problem to be solved. An intrinsic property of phages is their specificity, which is beneficial to minimize the disruption of normal microflora. However, such tight specificity makes it difficult to develop phages with broad antibacterial spectra for durable, off-the-shelf phage cocktail therapeutics.

### SUMMARY OF THE INVENTION

One approach is to prepare a phage cocktail that comprises various phages targeting a broad range of pathogenic bacteria. When phages in a phage cocktail encounter a pathogen that is refractory to phage infection, a new phage that can infect the resistant bacteria is generated by using a directed in vitro evolution approach (the Appelmans protocol). In a phage cocktail according to the “Appelmans protocol”, one or more phages adapt to overcome phage resistance of a bacterium (“phage training”), which might be explained by genetic mechanisms, spontaneous mutations as well as genetic recombination between phages. Using this approach, it is possible to develop phage clones with broader host ranges against a panel of global, diverse MDR clinical strains such as ESKAPEE pathogens.

Here it is disclosed to employ directed evolution in vitro to generate phage variants with broader host ranges for improved therapeutic cocktails for the goal of obtaining phages having broader host ranges for the development or improvement of therapeutic phage cocktails. For that purpose, a directed in vitro evolution approach (the Appelmans protocol) was employed to develop Pseudomonas phages as well as Klebsiella phages against panels of global, diverse MDR clinical strains.

In this disclosure, it is demonstrated that a directed in vitro evolution approach can yield phages with expanded or altered host ranges. Such phage clones with an expanded host range can provide powerful additions to a therapeutic phage cocktail design by reducing the number of phages required for a broad host range of bacterial strains, in a currently available phage cocktail, which provides better activity directed at phage-resistant strains. Using this method, phages trained, i.e., evolved against P. aeruginosa or K. pneumoniae, were obtained, 4 as Pseudomonas phages and 3 Klebsiella phages.

Then, the phage clones with expanded host ranges were isolated and characterized. Sequence analysis identified single nucleotide polymorphisms (SNPs) and areas of recombination in the evolved phage clones relative to parental phages.

The ability of phages to cause bacterial lysis in expanded host range strains was stably maintained for some phages. Among them, Pseudomonas phage 20176 4-2 was selected because it was one of the trained phages with stably expanded host range and compatibility with top performing phages. A Pseudomonas phage cocktail previously developed, WRAIR_PAM3, covers 83% of a 100-strain diversity panel, and a Klebsiella cocktail, KPM1, covers only 50% of a 100-strain diversity panel. The selected phage clone, 20176-4-2, replaced two phages in the 6-phage cocktail WRAIR_PAM3 to make a 5-phage cocktail WRAIR_PAM3T. This replacement expanded the cocktail activity to 85%, reduced the number of cocktail components (which simplifies cocktail manufacturing), added a phage binding to a different bacterial receptor (which is important to reduce phage resistance) and improved antibacterial therapeutic efficacy in a mouse model of wound infection.

Based on this discovery, here is provided a new method for phage training employing a directed in vitro evolution and pharmaceutical compositions comprising the phages obtained using said method.

### BRIEF DESCRIPTION OF THE DRAWINGS

FIG. 1 shows the basic steps of the Appelmans protocol.

FIG. 2 is a flowchart showing the phage training protocol used in this disclosure from start to finish. (a) Pooled lysates were filter-sterilized and then used to seed plates for the next round of training. Lysates were checked on agar plates every five rounds to check for plaque formation. Following isolation and purification of trained phage clones, the stability of host range expansion was assessed via serial propagation against a single permissive host. (b) Illustration of the method for testing stability of host range expansion maintained after phage training. Phage clones were propagated on a phage-permissive host, and five serial propagations were conducted. The host range of passaged phage clones isolated from the collected plaques were compared with the parental phages.

FIG. 3 shows plaque morphology of isolated phages using KB-Pa (Pseudomonas phage training panel) and KB-Kp (Klebsiella phage training panel), following purification from their respective isolation host strains (MRSN20176, MRSN15882, and MRSN27989). The phages displayed lytic activity of 2+ or 3+ and displayed ‘pinprick’ to small plaque sizes (0.5-2 mm).

FIG. 4 shows lytic profiles of KB-Pa (Pseudomonas phage training panel)-evolved P. aeruginosa phage clones isolated from training strain MRSN20176 compared with parental phages. (a) Lysis scoring reflects the quality of plaque formation when plated against 100 P. aeruginosa strains in a global diversity panel [Lebreton F, Snesrud E, Hall L, Mills E, Galac M, Stam J, Ong A, Maybank R, Kwak Y I, Johnson S, Julius M, Ly M, Swierczewski B, Waterman P E, Hinkle M, Jones A, Lesho E, Bennett J W, McGann P. A panel of diverse Pseudomonas aeruginosa clinical isolates for research and development. JAC Antimicrob Resist. 2021 Dec. 10; 3(4):dlab179. doi: 10.1093/jacamr/dlab179. PMID: 34909689; PMCID: PMC8664536]. Host range coverage was determined by replicative lysis (2+ activity and higher); expansion was determined relative to the best performing parental phage, KEN1. (b) Parental phages and trained phage clones tested with lytic activity.

FIG. 5 shows lytic profile of KB-Kp (Klebsiella phage training panel)-evolved K. pneumoniae phage clones isolated from strain MRSN15882 and MRSN27989 compared with their parental phages. (a) Lysis scoring reflects quality of lysis when tested against 100 K. pneumoniae strains in a global diversity panel (Lebreton et al.). Host range coverage was determined by replicative lysis (2+ activity and higher), and expansion was determined relative to the best performing parental phage, KEN39. (b) Parental phages and trained phage clones tested with lytic activity.

FIG. 6(a) shows lytic profiles of clones collected following stability testing of the top performing evolved clones. Stability of host range expansion was observed for phage clones collected from MRSN20176, with clones derived from phage 20176-4 gaining coverage, and clones derived from phages 20176-5, phage clones 20176-6-2, 20176-6-3, 20176-7-2, and 20176-7-3 retaining their expanded host range. FIG. 6(b) is the list of tested Pseudomonas phage clones.

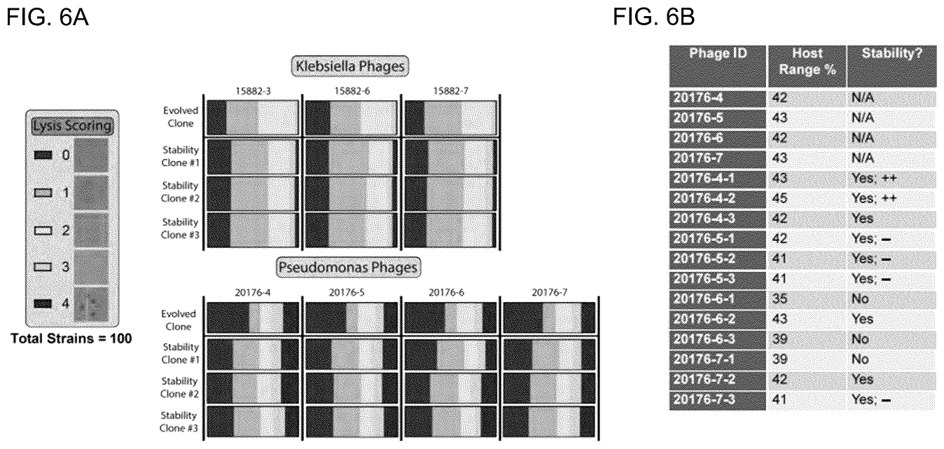

FIG. 7 shows that clone 20176-4-2 robustly kills infection/challenge strains of P. aeruginosa. (a) Lytic effect comparison of phage clone 20176-4-2 on PAO1 and PAO1::lux strains. (b) Phage clone 20176-4-2 is better at inhibiting PAO1::lux growth than each of three parental phages (KEN1, KEN10, and AFR43)

FIG. 8 shows timeline of an animal experiment in a mouse dorsal wound infection model.

FIG. 9 shows therapeutic effect of PAM3T treatment in a mouse wound infection model. Phage cocktail PAM3T compared with antibiotic treatment (CAZ BID: 410 mg/kg ceftazidime) reduced bacterial luminescence in wounds. The most rapid and robust antibacterial effect was observed for the phage cocktail PAM3T and antibiotic combo-treated group.

FIG. 10 summarizes the therapeutic effect of PAM3T treatment in a mouse wound infection model of FIG. 9 in graphs. (a) mouse survival rates, (b) wound healing (mean time to wound closure). (c) table with mean time to wound closure data.

FIG. 11 shows the result of Appleman training round by round in MDR P. aeruginosa strains.

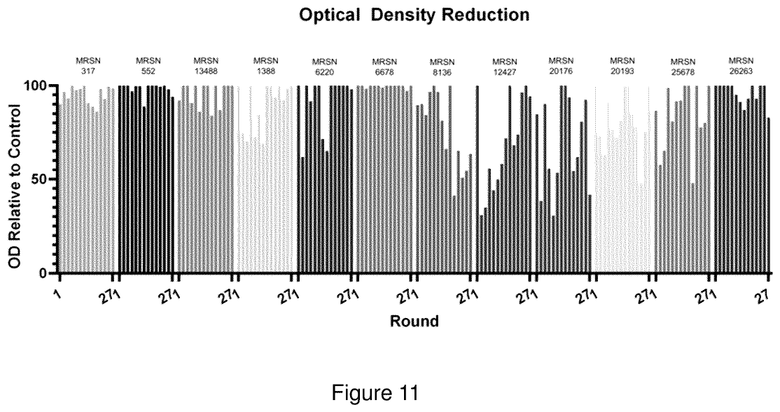

### DETAILED DESCRIPTION

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present disclosure. It will be apparent, however, to one skilled in the art that the present invention may be practiced without these specific details.

### A. Definitions

Embodiments of materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used.

It is also to be understood that the terminology used herein is for the purpose of describing particular embodiments only, and it is not intended to be limiting.

As used herein and in the appended claims, the singular forms “a,” “an,” and “the” include plural references unless specifically stated otherwise.

Other than in the operating examples, or where otherwise indicated, all numbers expressing quantities of ingredients or reaction conditions used herein should be understood as modified in all instances by the term “about.” The term “about” generally refers to a range of numbers that one of skill in the art would consider equivalent to the recited value (i.e., having the same function or result). In a specific embodiment, the term “about” includes a stated numerical value as well as a value that is +/−15% of the stated numerical value. For example, about 5.75 M includes 5.75 molar as well as 6.61 M and 4.89 M, and all 1/10 values in between. In many instances, the term “about” may include numbers that are rounded to the nearest significant figure.

As used herein, the term “bacteriophage” refers to bacterial viruses with a DNA or RNA genome, which is usually protected by a membrane or protein shell (capsid), and long (contractile or non-contractile) or short non-contractile tail with fibers responsible for binding to a bacterial cell. Bacteriophages infect bacteria either to replicate to large numbers and cause the cell to lyse (lytic infection) or, in some cases, to integrate into the bacterial genome without killing the host bacteria (lysogenic infection). In the lytic infection, viral DNA and proteins are rapidly synthesized and packaged into virus particles, leading to the lysis (destruction) of the host bacteria and then sudden release of progeny virus particles (virions). In contrast, in the lysogenic infection, the phage DNA is inserted into the host bacterial chromosome (prophage state), replicated together with bacterial chromosome, and transmitted to daughter cells. Lysogeny can sometimes significantly affect the host (lysogenic conversion). Under certain conditions causing DNA damages, the prophage is excised and initiates a lytic cycle, leading to the formation of progeny viruses and lysis of the host. (Fortier L C, Sekulovic O. Importance of prophages to evolution and virulence of bacterial pathogens. Virulence. 2013 Jul. 1; 4(5):354-65.) As used herein, the term “lytic activity” refers to phage activity leading to cell lysis of a target bacteria as determined by a plaque assay.

As used herein, the term “plaque assay” refers to phage titration, which is a process for isolation of phage clones and quantitation of the number of viable phage particles in a sample, which is presented as plaque-forming units (pfu) per unit volume, indicating active phage particle numbers in a unit volume of a phage suspension. For plaque assay, 10-fold serial dilutions of a phage sample are carried out through 10⁻¹⁰, depending on the phage titer; mixing each 10-fold serial dilution of the phage sample with aliquots of phage-sensitive bacteria suspended in bacterial culture medium; incubating the mixture of phage and bacteria for 10 minutes to allow for adsorption (attachment) of the phage to the bacteria; preparing tubes containing top agar (0.7% agar in bacterial culture medium) warmed at the 47° C. water bath; adding certain volume of phage and bacteria mixture (usually 100 μL) into a top agar tube, carefully mixing, pouring onto the appropriately labeled bottom agar plate (1.5% agar in bacterial culture medium), one plate for each phage dilution; allowing the top agar to solidify (about 5-10 minutes); and inverting the plates and incubating the plates at 37° C. Phage-infected and lysed bacteria release phages that infect and lyse the surrounding bacteria in the top layer, and multiple rounds of infection and lysis continue until the area of the infected bacteria on the plate is cleared, which appears as individual holes, or plaques, in an otherwise confluent bacterial lawn. Since each plaque is caused by a single viable phage when there are much less phages than bacteria, counting the number of plaques can be assumed as the number of plaque-forming units in the original suspension.

In the specification, the term “phage cocktail” means a phage mixture comprising two or more phages of the invention, or variants or progeny thereof, each of which have been isolated from the environment from which they were originally found or have been produced by means of a technical process such as genetic engineering or serial passage techniques.

As used herein, the term “plaque isolation” refers to recovering one single plaque with e.g., an Eppendorf tip and subsequently suspending it in a small volume (e.g., 500 μL or 1.5 mL in a tube) of phage buffer, with subsequent chloroform treatment or ultrafiltration to remove live and dead bacterial cells. This can be applied to a small volume (few mL) bacterial culture or spread on the surface of a host agar plate for bacterial lysis.

As used herein, the term “phage propagation” refers to amplifying phages by starting from a single plaque, from phage suspension, or floating agar in a plate that has confluent lysis. Bacterial lysis can be done in a liquid bacterial host culture or on a plate by using the double agar layer technique with top and bottom agar. When using a plate for bacterial lysis by phages, the bottom agar has usual agar concentration (1-1.5%) containing culture nutrients for the host; the top layer has the agar concentration (0.3% to 0.75%) containing the host bacteria mixed in this “soft agar” to form a homogenous thin layer of bacterial lawn. The phages can be dropped onto the top layer or added already into the melted soft agar cooled to 47° C. Then, the plate is incubated by shaking carefully on a plate shaker for few hours. The soft agar layer with confluent lysis is scraped off, centrifuged for removing agar and bacterial cell debris. The supernatant is filtered through a 0.45 m filter, and then through a 0.2 m filter to get a phage stock solution. When using liquid bacterial culture for bacterial lysis, the phages are added to the bacterial culture of a log phase OD₆₀₀ being cultured in a shaking incubator at 37° C., letting the bacteria and phages grow together until lysis occurs, i.e., the culture media becomes almost clear or transparent. This process needs to be observed because some phage-resistant bacterial cells may overgrow and make the culture turbid again. (www.dsmz.de/fileadmin/user_upload/Collection_allg/DSMZ_Cultivation_of_Phages.pdf) As used herein, the term “antibiotics” refers to antimicrobial substance active against bacteria for the treatment of bacterial infections, which usually either kill or inhibit the growth of bacteria. General antibiotics can be classified into to: (1) bacterial cell wall or membrane synthesis inhibitors, e.g., penicillins and cephalosporins; (2) protein synthesis inhibitors (e.g., macrolides, tetracyclins); and (3) DNA synthesis inhibitors (e.g., fluoroquinolones gyrase inhibitors and sulfa antibiotics inhibiting bacterial folic acid synthesis). Standard or traditional antibiotic agents include, but are not limited to: (1) cell wall or membrane synthesis inhibitor antibiotics, e.g., penicillins or beta-lactam compounds: pencillins (e.g., penicillin G, penicillin V, isoxazolyl penicillins, oxacillin, cloxacillin, flucloxacillin, dicloxacillin, nafcillin, methicillin, ampicillin, amoxicillin, piperacillin, ticarcillin, carbenicillin, aminopenicillins, carboxypenicillins, ureidopenicillins, temocillin, azlocillin, mezlocillin, mecillinam); cephalosporins and cephamycin: (e.g., cefazolin, cephalexin, cephradine, cefadroxil, cefuroxime, cefaclor, cefamandole, cefonicid, cefprozil, ceforanid, cefoxitin, cefmetazole, cefotetan, cefoperazone, cefotaxime, ceftazidime, ceftriaxone, cefepime, ceftaroline fosamil, cephalothin, cephapirin, cefpodoxime, ceftibuten, cefdinir, ceftizoxime, ceftriaxone, cefepime, cefditoren, cefixime, ceftibuten, cefacetrile, cefaloglycin, cefalonium, cefaloridine, cefatrizine, cefazaflur, cefazedone, cefroxadine, ceftezole, cefuzonam, cefcapene, cefdaloxime, cefdinir, cefditoren, cefetamet pivoxil, cefmenoxime, cefteram, ceftiofur, cefoperazone, ceftazidime latamoxef, cefclidine, cefluprenam, cefoselis, cefozopran, cefpirome, cefquinome, ceftobiprole, flomoxef (or oxa-1-cephamycin), ceftobiprole); carbacephems (e.g., loracarbef); carbapenems (e.g., biapenem, doripenem, ertapenem, imipenem, imipenem-cilastatin, meropenem, tebipenem pivoxil, faropenem, panipenem/betamipron, razupenem (PTZ-601), thienpenem (thienamycin)); monobactams (e.g., aztreonam, tigemonam, nocardicin A, or tabtoxinine β-lactam); beta-lactamase inhibitors (e.g., clavulanic acid, sulbactam, tazobactam); glycopeptides (e.g., vancomycin, teicoplanin); lipoglycopeptide (e.g. oritavancin, dalbavancin and telavancin); daptomycin, fosfomycin, bacitracin, cycloseine, isoniazid; polypeptide antibiotics (e.g., polymyxin B, or polymyxin E (colistin)); (2) antibiotics targeting bacterial ribosome subunits, the 30S and the 50S subunits; dactinomycin (or actinomycin D), chloramphenicol; tetracyclines (e.g., tetracycline, chlortetracycline, doxycycline, oxytetracycline, demeclocycline, lymecycline, meclocycline, methacycline, minocycline, rolitetracycline, or tigecycline); macrolides (e.g., erythromycin, clarithromycin, azithromycin, fidaxomicin, telithromycin, carbomycin A, josamycin, kitasamycin, midecamycin/midecamycin acetate, oleandomycin, solithromycin, spiramycin, troleandomycin, tylosin, or roxithromycin, dirithromycin, roxithromycin); aminoglycosides: (e.g., streptomycin, rhodostreptomycin, kanamycin, neomycin, amikacin, gentamicin, netilmicin, tobramycin, paromycin, apramycin) apramycin, plazomicin, arbekacin, and streptomycin); lincosamides (e.g., lincomycin, pirlimycin and clindamycin); ansamycins: geldanamycin, naphthomycin, rifamycins (e.g., rifampicin (or rifampin), rifabutin, rifapentine, rifalazil, or rifaximin); quinupristin-dalfopristin, mupirocin, streptogramins (e.g., streptogramin A, streptogramin B), oxazolidinones (e.g., linezolid, tedizolid), spectinomycin; (3) DNA replication inhibitor antibiotics: fluoroquinolones and quinolones (e.g., nalidixic acid, norfloxacin, ciprofloxacin, levofloxacin, gatifloxacin, moxifloxacin, gemifloxacin, lomefloxacin, ofloxacin, pefloxacin, moxifloxacin, rosoxacin, enoxacin, fleroxacin, nadifloxacin, rufloxacin, balofloxacin, grepafloxacin, pazufloxacin, sparfloxacin, temafloxacin, tosufloxacin, clinafloxacin, garenoxacin, stifloxacin, trovafloxacin, prulifloxacin, cinoxacin, flumequine, oxolinic acid, piromidic acid, pipemidic acid); sulfonamide antibiotics (e.g., sulfacytine, sulfisoxazole, sulfamethizole, sulfadiazine, silver sulfadiazine, sulfamethoxazole, sulphapyridine, sulfadoxine, sulfathalidine, sulfacetamide sodium, mafenide, co-trimoxazole, sulfasalazine, sulfanilamides, sultiame, sulfadimethoxine; pyrimidines (e.g., trimethoprim, pyrimethamine, and more than 200 drugs; https://go.drugbank.com/categories/DBCAT000349); others (pyrazinamide, ethambutol, streptomycin, ansamitocin); and any combination thereof can be used.

As used herein, the term “multidrug resistance” refers to antibacterial resistance which happens when bacteria develop the ability to defeat the drugs designed to kill them. That means bacteria are not killed and continue to grow. CDC typically uses this term to refer to an isolate that is resistant to at least one antibiotic in three or more antibiotics classes. Multidrug resistance mechanisms fall into four main categories: (1) limiting uptake of a drug; (2) modifying a drug target; (3) inactivating a drug; (4) active drug efflux. There are two main ways that bacterial cells can acquire antibiotic resistance. One is through mutations that occur in the DNA of the cell during replication. The other way that bacteria acquire resistance is through horizontal gene transfer through plasmids or transposons coding for resistance to a specific agent. Examples of bacteria resistant to antibiotics are methicillin-resistant Staphylococcus aureus (MRSA), vancomycin-resistant Enterococcus (VRE), multi-drug-resistant Mycobacterium tuberculosis (MDR-TB) and carbapenem-resistant Enterobacteriaceae (CRE) gut bacteria. Generally, MDR is defined as non-susceptibility to ≥1 agent in ≥3 antimicrobial categories, extensively drug-resistant (XDR) as non-susceptibility to ≥1 agent in all but ≤2 categories, and pandrug-resistant (PDR) as non-susceptibility to all antimicrobial agents listed. (Magiorakos A P et al., Multidrug-resistant, extensively drug-resistant and pandrug-resistant bacteria: an international expert proposal for interim standard definitions for acquired resistance. Clin Microbiol Infect. 2012 March; 18(3):268-81.) As used herein, the term “subject” or “patient” encompasses the whole phyla of animal kingdom, including Arthropoda, Mollusca, Chordata, for example, crab, lobster, shrimp, squid, oysters, clams, mussels, or other shellfish, eel, fishes, amphibians, reptiles, birds, and mammals that include domestic-, farm-, zoo- and wild animals, primates, and humans, who may be suffering from P. aeruginosa or K. pneumoniae infection, particularly an infection caused by non-MDR or MDR P. aeruginosa or K. pneumoniae strains in the lung, urogenital organ, skin or blood stream. For example, the subject has P. aeruginosa or K. pneumoniae infection after lung transplant or is suffering from pneumonia, cystic fibrosis, bronchiectasis, bladder infection (cystitis), kidney infection (pyelonephritis), skin infection (cellulitis, burn wounds), and/or sepsis. In addition, the subject may include mushrooms, algae, or plants that can be infected with non-MDR or MDR P. aeruginosa or K. pneumoniae strains.

As used herein, the term “treatment” in the context of pharmacological or medical meaning refers to intervention of disease, disorder, condition or symptoms to obtain a desired physiologic effect. “Treatment” includes: inhibiting the disease, disorder, condition, or symptoms thereof, such as, arresting its development or progression, and also includes relieving, alleviating, or ameliorating the disease, disorder, condition, or reducing one or more symptoms thereof, such as, for example, reducing infection.

As used herein, the term “administering” refers to introducing an agent to a subject, and can be performed using any of the various methods for drug or composition delivery known to those skilled in the art. Routes of administering include, but are not limited to oral administration, parenteral administration (subcutaneous (SC/SQ: <1 mL), intravenous (IV: 1-20 mL), intradermal (ID: <0.2 mL), intramuscular (IM: <4 mL), intraperitoneal (IP), intraarterial, intracardiac, intraarticular, and intraspinal injection), rectal administration by way of suppositories or enema, local/topical administration directly into or onto a target tissue, nasal administration (nebulizer, nasal spray, inhalation), or administration by any route or method that delivers a therapeutically effective amount of the drug or composition to the cells or tissue to which it is targeted.

As used herein, the term “therapeutically effective amount” refers to an amount effective, at dosages and for periods of time necessary, to achieve the desired therapeutic results. A therapeutically effective amount is also one in which any toxic or detrimental effects of compounds of the invention are outweighed by the therapeutically beneficial effects.

As used herein, the term “composition” refers to a pharmaceutical composition, meaning a mixture of substances suitable for administering to an individual, which includes one or more pharmaceutically active ingredients. For example, a pharmaceutical composition may comprise a certain amount of phage particles in solution or lyophilized dry powder as well as suitable pharmaceutical excipients.

As used herein, the term “excipient”, in the context of pharmaceuticals, refers to any substances other than the active ingredient or agent, contained in pharmaceutical dosage forms. The excipients are considered as inert substances, i.e., they do not have any active role in therapeutics, but they can be used to support the process to produce an effective product. Examples of excipients are active pharmaceutical ingredient excipients, binder excipients, capsule shell excipients, carrier excipients, coating systems excipients, controlled release excipients, diluent excipients, disintegrant excipients, effervescent system excipients, emulsifier excipients, film former excipients, flavor excipients, high-functionality excipients, lipid excipients, lubricant excipients, modified release excipients, penetration enhancer excipients, permeation enhancer excipients, pH modifier excipients, plasticizer excipients, preservative excipients, sachet filling excipients, solubilizer excipients. solvent excipients, surfactant excipients, sustained release excipients, taste masking excipients, thickener excipients, viscosity modifier excipients, blending excipients, filler excipients, compaction excipients, direct compression excipients, dry granulation excipients, hot melt extrusion excipients, wet granulation excipients, rapid release agent excipients, film formation excipients, increased bioavailability excipients, dispersion excipients, solubility enhancement excipients, stabilizer excipients, capsule filling excipients, powder blends excipients, tablet compressibility excipients, etc. (https://www.americanpharmaceuticalreview.com/25335-Pharmaceutical-Raw-Materials-and-APIs/25283-Pharmaceutical-Excipients/) As used herein, the term “dosage” refers to the administering of a specific amount, number, and frequency of doses over a specified period of time, and the term “dose” refers to a specified amount of medication taken at one time. A “dosage regimen” refers to the number of doses of a drug, medication, or an agent that a patient is supposed to take (or to be administered) over a specified period of time, and the individual doses that comprise the regimen are usually scheduled.

As used herein, the term “dosage form” refers to a pharmaceutical preparation in which a specific mixture of active ingredients of a drug and inactive components (excipients) are formulated in a particular shape or form to facilitated administration and accurate delivery of active ingredients, and/or to be presented in the market. Solid dosage forms include powders, granules, capsules, tablets, cachets, pills, lozenges, gummies, suppositories. Semi-solid dosage forms include ointment, creams, paste, gels, poultices. Liquid dosage forms include collodions, droughts, elixirs, emulsions, suspension, enemas, gargles, linctuses, lotion, liniments, mouth washes, nasal drop, paints, solutions, syrups. Gaseous dosage forms include aerosols, inhalations, and sprays. (https://thepharmapedia.com/pharmaceutical-dosage-form-pharmaceutics/pharmacy-notes/)

As used herein, the terms “administer” and “administration,” when used with respect to a drug or an agent (including antibodies and antibiotics), means providing the drug or agent to a subject using any of the various methods or delivery systems for pharmaceutical compositions known to those skilled in the art. For example, the administration of the drug can be oral, nasal, parental, topical, ophthalmic, or transdermal delivery of the drug in the form of solid, semi-solid, lyophilized powder, or liquid dosage forms.

As used herein, the terms “co-administration” refers to the administration of a first active agent before, concurrently, or after the administration of a second active agent such that the biological effects of the two (or more) agents overlap.

As used herein, the term “nucleic acid” refers to any polyribonucleotide or polydeoxyribonucleotide that may be unmodified RNA or DNA or modified RNA or DNA. Thus, for instance, nucleic acid as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or double-stranded, or a mixture of single- and double-stranded regions.

As used herein, “sequence identity” or “identity” in the context of two nucleic acid- or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

### B. Overview

Phages are potentially powerful therapies for drug-resistant bacterial infections. The high specificity of phages makes it complicated to develop phage therapeutics. In certain embodiments, directed evolution (e.g., Appelmans protocol) was used to generate phages with expanded host ranges.

As used herein, the term “Appelmans protocol” refers to an empirical liquid method of phage titration developed in the 1920s by Appelmans, whose working mechanism(s) has not been clarified. (Appelmans, R. Le dosage du Bacteriophage. Compt. Rend. Soc. Biol. 1921, 85, 1098; Chanishvili, N. & Sharp, R. Eliava Institute of Bacteriophage, Microbiology and Virology, Tbilisi, Georgia. A Literature Review of the Practical Application of Bacteriophage Research; Eliava Foundation: Tbilisi, Georgia, 2009; and Burrowes B H, Molineux I J, Fralick J A. Directed in Vitro Evolution of Therapeutic Bacteriophages: The Appelmans Protocol. Viruses. 2019 Mar. 11; 11(3):241).

In certain embodiments, a method utilizing phage evolution in vitro to generate therapeutic phages having expanded lytic host ranges is provided. Briefly, phages, which belong to the same genus but are complement to each other in their antibacterial/lytic activity, are selected and mixed to make a phage cocktail. The phage cocktail is repeatedly grown on a set of bacterial strains, including both phage-resistant and phage-susceptible host bacteria in multiple rounds (“training”) until the phages in the cocktail have evolved so as to lyse the phage-resistant strains. Phage clones showing lytic activity in an expanded host range are collected and purified, and then their DNA is isolated, sequenced and compared with control/parent phages (“untrained”).

As a proof-of-principle experiment aimed at examining the mechanism of host-range expansion, a 96-well plate format Appelmans protocol was designed, using a 3-phage cocktail against P. aeruginosa and 4-phage cocktail against K. pneumoniae. The selected phages belong to the same genus, respectively, but are complement to each other in their antibacterial activity. For phage training, a panel suite comprising MDR 17 clinical isolates and one phage susceptible strain of P. aeruginosa and a panel suite comprising 10 MDR strains and one phage susceptible strain of K. pneumoniae were prepared. The phages are incubated with both phage-resistant and phage-susceptible host bacteria through multiple rounds.

After several (e.g. 3-20) rounds of selection, individual phages were isolated that could grow on a pan-resistant strain for each bacterial species. Next, the selected phages were tested with 100 globally diverse strains of the bacteria, and clones with extended activity were collected, and purified.

Then, phage DNA is isolated, sequenced and compared with “parent” phages. These phage clones clearly differ from natural phages. Sequence analysis demonstrated SNPs and recombination events occurred in predicted tail fiber genes, which is the area for recognition of the host bacteria, and this likely drove host range expansion, Generally, phage tails are composed of repetitive protein subunits that form a rod-like structure with a globular domain at the distal end, and the structure, number of subunits, and location of the tail fibers differ among the phage classes. (Filik K, et al., Bacteriophage Tail Proteins as a Tool for Bacterial Pathogen Recognition-A Literature Review. Antibiotics (Basel). 2022 Apr. 21; 11(5):555) After stability test, one of the best phage clones with stable expanded activity was selected and used in therapeutic cocktails. One “trained” phage clone, 20176-4-2, replaced two phages in 6-phage cocktail PAM3. The new 5-phage cocktail PAM3T has broader activity, more diverse components and easier to manufacture. Therapeutic effect of PAM3T treatment was tested in a mouse wound infection model. When phage cocktail PAM3T was compared with antibiotic, it was more effective in reducing bacterial infection in wounds. The most rapid and robust antibacterial effect was observed for the combo-treated group.

Phage training via the Appelmans protocol enabled host range expansion in phages targeting MDR bacterial strains. For some of the evolved P. aeruginosa phage clones, expansion in host range coverage was stable even after passages on a single permissive host. These phages show promise as possible therapeutic candidates.

As disclosed herein, it was demonstrated that this approach works both for Pseudomonas aeruginosa and Klebsiella pneumoniae phages. The approach works well on two genera of P. aeruginosa podophages, Bruynoghevirus and Phikmvvirus (data not shown), as well as myophages of K. pneumoniae (genus Jiaodavirus), thus showing that recombination-stimulating approach works universally on any number of phages. In addition, it was demonstrated that a phage obtained via directed evolution can be incorporated in a therapeutic cocktail, significantly improving it (reducing the number of components, expanding activity and increasing phage diversity, which is important to address a problem of phage resistance)—this cocktail, PAM3T, showed the highest efficacy among all our P. aeruginosa phage cocktail iterations in a mouse wound infection model; and incorporation of a “trained” K. pneumoniae phage in a 5-phage cocktail expanded its activity from 50% to 81%.

In a publication by another group, a mix of three P. aeruginosa phages was used for the Appelmans protocol, two of which belonged to the same genus (Litunavirus), and a recombinant clone of these two with slight expansion of activity was isolated; proving the presence of a non-homologous phage did not help in recombination and selection of phage progeny with much improved properties.

The phages generated by in vitro directed evolution in a phage cocktail comprising 3-4 taxonomically similar phages as described herein have expanded host range and/or activity against previously phage resistant strains. The evolved phages can stably maintain changes in host range, and the selected “trained” phage as an example was efficacious as therapeutics in mixture with other phages. Accordingly, the method embodiments described herein can be used for “training” phages against various MDR strains of various bacterial species, e.g., ESKAPEE.

### C. Embodiment Examples

The Appelmans protocol is a method by which phages in a phage cocktail can expand their host range without addition of new genetic information, solely depending on in vitro evolution.

In some embodiments, a method of the modified protocol is employed for expanding the antibacterial activity of phages, wherein the method comprises steps of:
i. mixing taxonomically similar phages with complimentary lytic activity;
ii. passaging mixed phages against a panel of phage-resistant bacterial clinical isolates;
iii. pooling lysates from wells showing reduction in bacterial growth and using them to seed subsequent rounds;
iv. periodically checking for plaque formation on phage-resistant strains;
v. isolating and purifying phage clones, sequencing and re-assessing host range; and
vi. assessing stability via serial propagation against single strain and re-assessing host range.

In certain embodiments, the modified protocol is employed to generate phages against MDR strains of P. aeruginosa or Klebsiella pneumoniae, using panel sets of MDR strains of the bacteria.

For the experiments in this disclosure, a panel set of P. aeruginosa strains for phage training comprises MDR strains comprising MRSN 317, 552, 1388, 2144, 6220, 6678, 8130, 8136, 8914, 12427, 13488, 16344, 20176, 20193, 25678, 26263, and 358800, and PAO1, a phage-susceptible P. aeruginosa strain; and a panel set of K. pneumoniae strains for phage training comprises MDR strains comprising MRSN 4759, 6778, 15687, 15882, 22232, 27989, 479404, 511348, 614201, and 681054, and 414780, a phage-susceptible K. pneumoniae strain. However, since MDR strains of both bacteria are continuously generated somewhere in the world, the bacterial strains in the panel can further include any newly appeared MDR strains.

In this disclosure, as an example of a phage-susceptible P. aeruginosa, PAO1 strain was used for phage propagation, and as an example of a pan-phage resistant P. aeruginosa, MRSN 20176 strain was used for plaque selection. Also, as an example of a phage-susceptible K. pneumoniae, MRSN414780 strain was used, and as an example of a pan-phage resistant K. pneumoniae, MRSN 15882 was used. However, other strains can be contemplated for the same purpose.

In addition, this method can be applied to developing phages against other bacterial species, especially MDR strains of ESKAPEE bacteria (Enterococcus faecium, Staphylococcus aureus, Klebsiella pneumoniae, Acinetobacter baumannii, Pseudomonas aeruginosa, Enterobacter spp. and E. coli) using Enterococci phages, Staphylococci phages, Acinetobacter baumannii phages, Enterobacter phages, and E. coli phages.

In the following, the aforementioned method is described in more detail. The steps are:
i. selecting at least two genetically similar phages, which have complementary lytic activity against a bacterial species, and determining the titer of each phage;
ii. mixing the phages at about the same ratio of each phage titer to make a phage cocktail to yield an input cocktail of 1×10⁵-1×10²⁰ pfu/mL, and optionally about 1×10¹⁰ pfu/mL;
iii. adding a certain volume of 10-20 MDR strains and a phage-susceptible strain of a bacterial species, which are individually cultured overnight in the wells of a multi-well plate filled with a certain volume of bacterial culture media, in one strain per one column (or row) manner;
iv. performing a serial 10-fold dilution of the phage cocktail of step ii (10⁰ to 10⁻⁷ or to 10⁻¹¹) in bacterial culture media;
v. adding each of the serial 10-fold dilutions of step iv into a row (or a column) of the wells containing the bacterial culture of step iii;
vi. incubating the plate(s) with shaking, optionally at 37° C. overnight;
vii. pooling the lysate from the bacterial culture wells showing reduced bacterial growth or bacterial lysis;
viii. clearing the pooled lysates, optionally by vortex mixing with CHCl₃ and centrifugation;
ix. repeating steps iii to viii with the cleared lysate at least 10 rounds;
x. while performing step ix, periodically, optionally every 5 rounds, testing plaque forming activity with the cleared lysate, using a double-layered agar plate with a pan-phage-resistant strain lawn;
xi. after final round, collecting individual phage plaques from the double-layered agar plate of step x;
xii. performing steps iii to vii with each collected individual phages;
xiii. selecting phages showing expanded host range when compared with parent phages;
xiv. testing the selected phages against genetically diverse strain global diversity set;
xv. selecting phages from the phages of step xiv, which show expanded host range when compared with parent phages in step xiv;
xvi. isolating and purifying the phage particles, and sequencing their DNAs;
xvii. testing phage stability with at least three clones of the selected phages; by re-testing them against the strain global diversity set to assess host range.

In this disclosure, as a non-limiting example of phages, three phages of Phikmvvirus genus are selected for the development of phages against MDR P. aeruginosa strain; KEN1 (SEQ ID NO: 28), KEN10 (SEQ ID NO: 29), and AFR43 (SEQ ID NO: 27); and five phages of Jiaodavirus genus are selected for the development of phages against MDR K. pneumoniae strains; KEN22 (SEQ ID NO: 30), KEN25 (SEQ ID NO: 31), KEN25-2, KEN37 (SEQ ID NO: 32), and KEN39 (SEQ ID NO: 33). However, other phages of the same genus can be contemplated if those phages can infect P. aeruginosa (or K. pneumoniae). Further, phages of other genera can be contemplated if they can infect P. aeruginosa (or K. pneumoniae). Furthermore, mixing P. aeruginosa phages (or K. pneumoniae phages) from more than one genus can be contemplated if they have similar genomes and complementary lytic activity against the bacteria. The number of phages to be mixed needs to be at least two, and thus five or more numbers of phages can be contemplated, which may present better gene recombination opportunity.

Also disclosed herein, 26 novel phages having new recombination genes were created. The following is the list of those phages.
1. A bacteriophage able to infect MDR strains of P. aeruginosa, wherein the phage is obtained according to claim 2, and wherein the phage comprises a nucleic acid sequence of SEQ ID NO:1 or a nucleic acid sequence of at least 89, 90, 95, or 97% sequence identity therewith, originated from phage plaque 20176-4.
2. A bacteriophage able to infect MDR strains of P. aeruginosa, wherein the phage is obtained according to claim 2, and wherein the phage comprises a nucleic acid sequence of SEQ ID NO:2 or a nucleic acid sequence of at least 89, 90, 95, or 97% sequence identity therewith, originated from phage plaque 20176-4-1.
3. A bacteriophage able to infect MDR strains of P. aeruginosa, wherein the phage is obtained according to claim 2, wherein the phage comprises a nucleic acid sequence of SEQ ID NO:3 or a nucleic acid sequence of at least 89, 90, 95, or 97% sequence identity therewith, originated from phage plaque 20176-4-2.
4. A bacteriophage able to infect MDR strains of P. aeruginosa, wherein the phage is obtained according to claim 2, and wherein the phage comprises a nucleic acid sequence of SEQ ID NO:4 or a nucleic acid sequence of at least 89, 90, 95, or 97% sequence identity therewith, originated from phage plaque 20176-4-3.
5. A bacteriophage able to infect MDR strains of P. aeruginosa, wherein the phage is obtained according to claim 2, and wherein the phage comprises a nucleic acid sequence of SEQ ID NO:5 or a nucleic acid sequence of at least 89, 90, 95, or 97% sequence identity therewith, originated from phage plaque 20176-5.
6. A bacteriophage able to infect MDR strains of P. aeruginosa, wherein the phage is obtained according to claim 2, and wherein the phage comprises a nucleic acid sequence of SEQ ID NO:6 or a nucleic acid sequence of at least 89, 90, 95, or 97% sequence identity therewith, originated from phage plaque 20176-5-1.
7. A bacteriophage able to infect MDR strains of P. aeruginosa, wherein the phage is obtained according to claim 2, and wherein the phage comprises a nucleic acid sequence of SEQ ID NO:7 or a nucleic acid sequence of at least 89, 90, 95, or 97% sequence identity therewith, originated from phage plaque 20176-5-2.
8. A bacteriophage able to infect MDR strains of P. aeruginosa, wherein the phage is obtained according to claim 2, and wherein the phage comprises a nucleic acid sequence of SEQ ID NO:8 or a nucleic acid sequence of at least 89, 90, 95, or 97% sequence identity therewith, originated from phage plaque 20176-5-3.
9. A bacteriophage able to infect MDR strains of P. aeruginosa, wherein the phage is obtained according to claim 2, and wherein the phage comprises a nucleic acid sequence of SEQ ID NO:9 or a nucleic acid sequence of at least 89, 90, 95, or 97% sequence identity therewith, originated from phage plaque 20176-6.
10. A bacteriophage able to infect MDR strains of P. aeruginosa, wherein the phage is obtained according to claim 2, and wherein the phage comprises a nucleic acid sequence of SEQ ID NO:10 or a nucleic acid sequence of at least 89, 90, 95, or 97% sequence identity therewith, originated from phage plaque 20176-6-2.
11. A bacteriophage able to infect MDR strains of P. aeruginosa, wherein the phage is obtained according to claim 2, and wherein the phage comprises a nucleic acid sequence of SEQ ID NO:11 or a nucleic acid sequence of at least 89, 90, 95, or 97% sequence identity therewith, originated from phage plaque 20176-6-3.
12. A bacteriophage able to infect MDR strains of P. aeruginosa, wherein the phage is obtained according to claim 2, and wherein the phage comprises a nucleic acid sequence of SEQ ID NO:12 or a nucleic acid sequence of at least 89, 90, 95, or 97% sequence identity therewith, originated from phage plaque 20176-7.
13. A bacteriophage able to infect MDR strains of P. aeruginosa, wherein the phage is obtained according to claim 2, and wherein the phage comprises a nucleic acid sequence of SEQ ID NO:13 or a nucleic acid sequence of at least 89, 90, 95, or 97% sequence identity therewith, originated from phage plaque 20176-7-2.
14. A bacteriophage able to infect MDR strains of P. aeruginosa, wherein the phage is obtained according to claim 2, and wherein the phage comprises a nucleic acid sequence of SEQ ID NO:14 or a nucleic acid sequence of at least 89, 90, 95, or 97% sequence identity therewith, originated from phage plaque 20176-7-3.
15. A bacteriophage able to infect MDR strains of K. pneumoniae, wherein the phage is obtained according to claim 2, and wherein the phage comprises a nucleic acid sequence of SEQ ID NO:21 or a nucleic acid sequence of at least 89, 90, 95, or 97% sequence identity therewith, originated from phage plaque 15882-3.
16. A bacteriophage able to infect MDR strains of K. pneumoniae, wherein the phage is obtained according to claim 2, and wherein the phage comprises a nucleic acid sequence of SEQ ID NO:22 or a nucleic acid sequence of at least 89, 90, 95, or 97% sequence identity therewith, originated from phage plaque 15882-3-1.
17. A bacteriophage able to infect MDR strains of K. pneumoniae, wherein the phage is obtained according to claim 2, and wherein the phage comprises a nucleic acid sequence of SEQ ID NO:23 or a nucleic acid sequence of at least 89, 90, 95, or 97% sequence identity therewith, originated from phage plaque 15882-3-2.
18. A bacteriophage able to infect MDR strains of K. pneumoniae, wherein the phage is obtained according to claim 2, and wherein the phage comprises a nucleic acid sequence of SEQ ID NO:24 or a nucleic acid sequence of at least 89, 90, 95, or 97% sequence identity therewith, originated from phage plaque 15882-3-3.
19. A bacteriophage able to infect MDR strains of K. pneumoniae, wherein the phage is obtained according to claim 2, and wherein the phage comprises a nucleic acid sequence of SEQ ID NO:25 or a nucleic acid sequence of at least 89, 90, 95, or 97% sequence identity therewith, originated from phage plaque 15882-6.
20. A bacteriophage able to infect MDR strains of K. pneumoniae, wherein the phage is obtained according to claim 2, and wherein the phage comprises a nucleic acid sequence of SEQ ID NO:26 or a nucleic acid sequence of at least 89, 90, 95, or 97% sequence identity therewith, originated from phage plaque 15882-6-1.
21. A bacteriophage able to infect MDR strains of K. pneumoniae, wherein the phage is obtained according to claim 2, and wherein the phage comprises a nucleic acid sequence of SEQ ID NO:27 or a nucleic acid sequence of at least 89, 90, 95, or 97% sequence identity therewith, originated from phage plaque 15882-6-2.
22. A bacteriophage able to infect MDR strains of K. pneumoniae, wherein the phage is obtained according to claim 2, and wherein the phage comprises a nucleic acid sequence of SEQ ID NO:28 or a nucleic acid sequence of at least 89, 90, 95, or 97% sequence identity therewith, originated from phage plaque 15882-6-3.
23. A bacteriophage able to infect MDR strains of K. pneumoniae, wherein the phage is obtained according to claim 2, and wherein the phage comprises a nucleic acid sequence of SEQ ID NO:29 or a nucleic acid sequence of at least 89, 90, 95, or 97% sequence identity therewith, originated from phage plaque 15882-7.
24. A bacteriophage able to infect MDR strains of K. pneumoniae, wherein the phage is obtained according to claim 2, and wherein the phage comprises a nucleic acid sequence of SEQ ID NO:30 or a nucleic acid sequence of at least 89, 90, 95, or 97% sequence identity therewith, originated from phage plaque 15882-7-1.
25. A bacteriophage able to infect MDR strains of K. pneumoniae, wherein the phage is obtained according to claim 2, and wherein the phage comprises a nucleic acid sequence of SEQ ID NO:31 or a nucleic acid sequence of at least 89, 90, 95, or 97% sequence identity therewith, originated from phage plaque 15882-7-2.
26. A bacteriophage able to infect MDR strains of K. pneumoniae, wherein the phage is obtained according to claim 2, and wherein the phage comprises a nucleic acid sequence of SEQ ID NO:32 or a nucleic acid sequence of at least 89, 90, 95, or 97% sequence identity therewith, originated from phage plaque 15882-7-3.

Since one of the goals of these new phage development is pharmaceutical application for the treatment of bacterial infection, certain embodiments pertain to pharmaceutical compositions comprising at least one of those phages, optionally 2, 3, 4, 5, 6, or 7 or more phages listed above. A pharmaceutical composition may comprise at least one of those phages mixed with or without other phages from the same genus or other genus or genera, which targets the same bacterial species or different bacterial species of the same genus or other genus or genera.

For the experiments in this disclosure, Pseudomonas phages from Phikmvvirus genus and Klebsiella phages from Jiaodavirus genus were used as unlimiting examples to demonstrate the method described above, but any Pseudomonas phages or Klebsiella phages can be used.

The members of Phikmvvirus genus (Lineage: Viruses; Duplodnaviria; Heunggongvirae; Uroviricota; Caudoviricetes; Autographiviridae; Krylovirinae), which are Pseudomonas phages, are listed on https://www.ncbi.nlm.nih.gov/Taxonomy/Browser/wwwtax.cgi?id=477967.

The members of Jiaodavirus genus (Lineage: Viruses; Duplodnaviria; Heunggongvirae; Uroviricota; Caudoviricetes; Straboviridae; Tevenvirinae), which are Klebsiella phages, are listed on https://www.ncbi.nlm.nih.gov/Taxonomy/Browser/wwwtax.cgi?id=1985325.

In a particular embodiment, a composition comprising other phages against P. aeruginosa in addition to phage of SEQ ID NO:3 is presented, wherein the phages are one, two, three, or four phages selected from genus of Pbunavirus (e.g., EPa11, EPa15, and/or Epa22), Nankokuvirus (Epa16 and/or Epa18), and/or Abidjan virus (e.g., Epa43). A preferred embodiment here is a composition comprising EPa11, EPa15, Epa16, Epa18, and phage of SEQ ID NO:3, which can be replaced with another phage or mixture of other phages of SEQ ID NO:1, 2, 4-16.

Such phage cocktail design can also be contemplated for a composition for the treatment of K. pneumoniae infection, wherein the composition comprises at least one phage selected from phages of SEQ ID NO:21-32.

Further, a phage cocktail comprising at least one phage from the P. aeruginosa phages and at least one phage from K. pneumoniae phages can be contemplated for the treatment of multiple infection, and phages against other bacterial species such as ESKAPEE, developed using the protocol described above, can be mixed with the phages of this invention.

### Formulations and Administration

For the treatment of bacterial infection, an effective amount of the phage or phage cocktail is administered to a patient through nasal (nebulizer, nasal or inhalation spray), parenteral (intravenous, intramuscular, intraperitoneal, subcutaneous, etc.) or topical route. Pharmaceutical composition for each administration route is formulated into a proper dosage form and comprises necessary excipients. For example, the phage or phage cocktail can be dissolved or suspended in saline or other buffers to be manufactured as a solution for nebulizer mist, nasal spray, or intravenous injection, and each of solutions for different route administration may have similar or different excipients.

In certain embodiments, the composition is formulated into a solution for parenteral administration.

In certain embodiments, the composition is formulated into a solution for nebulizer.

In certain embodiments, the composition is formulated into a solution or aerosol for nasal spray.

In certain embodiments, the composition is formulated into aerosol or dry powder for inhalation.

In certain embodiments, the composition is formulated into a lotion, cream, gel, an emulsion, ointment, or dry powder for topical administration.

In addition, such compositions may further comprise pharmaceutically acceptable excipients for nasal, parenteral, or topical administration. As used herein, the term “excipients” refers to substances that are added to therapeutic products to improve stability, bioavailability, and manufacturability. Examples of excipients are listed below.

For example, parenteral administration, the composition may comprise lyoprotectants (e.g., human albumin, lactose monohydrate, maltose/trehalose/sucrose, mannitol, dextran, inulin, fructose), micro-encapsulating agents (e.g., aliphatic polyester such as polyglycolide, polylactide, and their copolymers, phospholipids/lecithin, phosphatidic acids, phosphoglycerol, phosphoserine, phosphorethanolamine, phosphocholine, PEGylated phospholipids, hydroxypropylcyclodextrin, Betadex sulfobutyl ether sodium), solubilizers and emulsifiers (e.g., N-methyl 2-pyrrolidone, PEG, polysorbates, polyoxyl 35 castor oil, polyoxyl-15-hydroxystearate, polyvinyl pyrrolidone, propylene glycol, sodium cholesteryl sulfate, sorbitan esters, poloxamer, 2-pyrrolidone, diacylglycerols, monglycerol), tonicity agents (e.g., dextrose, glycerin, mannitol, NaCl, KCl, sorbitol/sorbitol solution), solvents and cosolvents (water miscible such as propylene glycol, PEG low molecular weight, glycerin, ethanol, 2-pyrrolidone, and N-methyl-2-pyrrolidone and water immiscible such as ethyl oleate, benzyl benzoate, vegetable oil, soybean oil, sesame oil, peanut oil, castor oil, almond oil, and cottonseed oil), viscosity-building agents (e.g., sodium carboxymethylcellulose (Na CMC), methylcellulose, gelatin, polyvinyl pyrrolidone), antioxidants (e.g., ascorbic acid, acetylcysteine, sodium ascorbate, sodium metabisulfite, sodium bisulfite, and tocopherol), chelating agents (e.g., EDTA), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), buffering agents (e.g., acetate, citrate, tartrate, phosphate, triethanolamine (TRIS) buffer).

For topical administration, the composition may comprise gelling agents (e.g., carbomer, carrageenan, chitosan, gelatin, gellan gum, pectin, ploxamer, poly(ethylene)oxide, polycarbophil, pullulan, HEC, HPMC, MC, alginates, Na CMC, xanthan gum, acacia, agar, guar gum, tragacanth, modified starch, povidone), humectants (e.g., corn syrup, glycerin, lactic acid, PEGs, propylene, glycol, sodium lactate, sorbitol, trehalose, xylitol), cream and ointment bases (e.g., cetostearyl alcohol, cetyl palmitate, fatty alcohols, hard fat, lanolin, lanolin alcohol, hydrogenated castor oil, mineral oil, petrolatum, glyceryl behenate, hard paraffin, soft paraffin, stearic acid, beeswax (white or yellow), carnauba wax, emulsifying wax, microcrystalline wax), solubilizers and emulsifiers (e.g., cocoylcaprylocaprate, decyl oleate, diethylene glycol monoethyl ether, dimethyl isosorbide, glyceryl monooleate, isopropyl myristate, medium chain triglycerides (MCT), octyldodecanol, oleyl alcohol, oleyl oleate, polyoxyethylene alkyl ethers, polyoxyethylene stearates, propylene glycol monocaprylate, propylene glycol monolaurate, sodium cetostearyl sulfate, lecithin, cyclodextrins, docusate sodium, glyceryl monostearate, hydrogenated vegetable/cottonseed/palm kernel oil, MCT, N-methyl-2-pyrrolidone, poloxamer, PEG, polysorbate, PEG castor oil derivatives, propylene glycol, polyoxyglycerides, sodium lauryl sulfate, sucrose esters), suspending agents and thickeners (e.g., magnesium aluminum silicate, MCC and Na CMC, propylene glycol alginate, acacia, carbomer, carrageenan, colloidal silicon dioxide, gellan gum, HEC, HPC, HPMC, maltitol, MC, pectin, PEGs, polyvinyl alcohol, povidone, Na CMC, sorbitol, sucrose, xanthan gum, tragacanth, gelatin, guar gum, kaolin, phospholipids), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), and stabilizers including pH modifiers (e.g., lactic acid, citric acid, tartaric acid, ascorbic acid, and their sodium salts), antioxidants referred to above, and chelating agent EDTA.

For nasal administration, the composition may comprise a suspending agent and thickener (e.g., colloidal carboxymethyl cellulose (CMC) and microcrystalline cellulose (MCC), MC, HPMC, pectin, PEGs), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), penetration enhancer (e.g., polysorbates, poloxamers, PEGs, propylene glycol, EDTA), tonicity agents (e.g., dextrose, glycerin, mannitol, NaCl, KCl, sorbitol/sorbitol solution), buffering agents (e.g., acetate, citrate, tartrate, phosphate, triethanolamine (TRIS) buffer). A composition for nebulizer solution may comprise buffering agents (e.g., phosphate buffer, citrate buffer), EDTA chelating agent, ethanol cosolvent, pH modifier (e.g., HCl, NaOH, sulfuric acid, tartaric acid, citric acid), preservatives (e.g., methyl paraben, propyl paraben, benzalkonium chloride), surfactant (e.g., polysorbate 20, polysorbate 80, sorbitan laurate), and tonicity agent (e.g., NaCl). A pressurized metered-dose inhaler composition may comprise propellants (e.g., hydrofluoroalkanes), surfactants (e.g., oleic acid, sorbitan trioleate, lecithin), pH modifier (e.g., citric acid), lubricant (e.g., PEG 1000, PEG 600), cosolvent (e.g., ethanol, glycerol), suspending agents (e.g., Povidone K25, K30). A dry powder—optionally lyophilized—inhaler composition may comprise carriers (e.g., lactose monohydrate), surfactants (e.g., dipalmitoyl phosphatidylcholine, distearoyl phosphatidylcholine), lubricant (e.g., magnesium stearate).

Further, a spray container, which is pressurized or not, containing the composition for nasal spray or inhalation spray can be contemplated, and the spray container optionally comprises an antibiotic or antibiotics in the dosage form of liposome, solution, or lyophilized dry powder, for example, liposomal ciprofloxacin, aztreonam solution, colistin solution or dry powder, tobramycin solution or dry powder. In a preferred embodiment, the spray container comprises ceftazidime. In other embodiments, methods of treating a subject having P. aeruginosa and/or K. pneumoniae infection are provided, and the methods comprise administering an effective amount of the composition described above, through nasal, parenteral, and/or topical route.

The composition comprising at least one phage described in this disclosure can be administered alone or co-administered with antibiotics, especially antibiotics against gram-negative bacteria. Antibiotics can be administered through oral, parenteral, topical, and/or nasal route together with the composition or separately before, after, or at the same time with the administration of the composition. In addition, some antibodies or fragments thereof against the surface antigens of the target bacteria may be contemplated for co-administration with the composition herein.

The subjects suitable for the treatment using the composition are patients with P. aeruginosa and/or K. pneumoniae infection in the lung, urinary tract, skin, and/or blood stream, and in particular patients who has P. aeruginosa and/or K. pneumoniae infection after lung transplant or are suffering from pneumonia, cystic fibrosis, bronchiectasis, bladder infection (cystitis), kidney infection (pyelonephritis), skin infection (cellulitis, burn wounds), and/or sepsis.

For excipients for other dosage forms including parenteral solution and topical gel, cream, paste, ointment, emulsion can be found in Remington: The Science and Practice of Pharmacy, 23rd Edition or higher version.

The composition can be administered once a day, twice a day, thrice a day for one day, couple of days, three days, four days, five days, six days, a week, two weeks, three weeks, a month or a longer period. Daily dose (pfu) is to be determined empirically, depending on the severity of P. aeruginosa and/or K. pneumoniae infection. For example, patients can be administered with 10⁵-10¹⁵ PFU, optionally 10⁹-10¹¹ PFU of a single phage or combination of at least two phages intravenously twice daily; or with the same dose by inhalational nebulization. Patients can also receive antibiotic treatment with the phage treatment against their own target isolate. Initial duration of phage treatment can be 6 months, but it can be shorter or longer courses of treatment directed by clinical and microbiologic responses. Phage regimens can be adjusted by the treating clinicians as needed based on tolerability to phage-containing pharmaceutical formulation and lab tests during phage treatment.

Although the main subject of this invention is human, any animals including both vertebrates and invertebrates, and even some mushrooms, algae and plants, which can be infected by P. aeruginosa or K. pneumoniae, can benefit from treatment with the phages listed above or phage cocktail comprising any of them. For example, the composition comprising the phages or phage cocktail against non-MRD or MRD strains of P. aeruginosa or K. pneumoniae can be scattered as dry powder or solution to the fish tank or aquaculture for oysters, clams, mussels, or other shellfish, or algae farm.

Considering the aspect of bactericidal effect of phages, the composition can also be developed into hygiene control products. For example, the composition can be utilized as a disinfectant for cleaning the surface of medical device and instruments, and/or hospital furniture or walls, by applying a liquid, aerosol, or powder composition to the surface directly or to wiping cloth.

### EXAMPLES

### Example 1. Materials and Methods

1.1. P. aeruginosa Phage Training

Bacterial Strains and Bacteriophages All bacterial strains, except PAO1 and PAO1::lux, used in this study are taken from diversity panels of clinical isolates prepared by the Multidrug-Resistant Organism Re-pository and Surveillance Network (MRSN). Bioluminescent strain P. aeruginosa PAO1::lux was engineered by insertion of the luciferase gene cassette (lux) in the PAO1 chromosome, (Engeman et al 2021). Strains were characterized for antibiotic suscepti-bility and phage susceptibility, (Table. 1). Antibiotic and phage susceptibility rankings are derived from how many phages or antibiotics can lyse or kill the strain. The susceptibility data comes from analysis of phages from our library lysing these strains. All bacterial strains were grown in Heart Infusion Broth (HIB) with shaking at rpm=200 and at 37° C. For all experiments, bacterial cultures were prepared fresh the day before planned experiments and grown overnight.

Bacteriophages used in this study were provided to our group from collaborating labs locating at AFRIMS (Bangkok, Thailand) or USMRD-A (Nairobi, Kenya). The phages belong to the PhiKMVvirus genus, (Table. 2). It was hypothesized that due to their close relatedness, these phages should be more able to undergo recombination. Phages stocks were prepared by propagating phages on host isolation strains. In brief, concentrated phage stock was added to bacterial culture at mid-log phase in propagation media (HIB+glucose, MgCl₂, and CaCl₂)) and allowed to incubate with the bacterial culture for 4-6 hours or until the culture was cleared, whichever came first. Cultures were then pelleted and phage lysate was filter-sterilized by passing through a 0.22 um syringe filter. Filtered phage lysates were used as the initial inoculum for the phage training. Phage lysates were stored, protected from light, at 4° C.

Strains were used in either one or two training panels, indicated by P1 or P2. All strains were selected on the basis of being highly phage-resistant broadly and resistant to all three selected phages specifically (AFR43, KEN1, and KEN10) except for PAO1. PAO1 was selected due to its high phage susceptibility including towards all three phages.

### Appelmans Training:

Appelmans training was conducted with minor modifications from the protocol described by Burrowes et al, [Burrowes B H, Molineux I J, Fralick J A. Directed in Vitro Evolution of Therapeutic Bacteriophages: The Appelmans Protocol. Viruses. 2019; 11(3):241. doi.org/10.3390/v11030241]. In brief, phage resistant bacterial clinical isolates were selected. They were grown overnight in HIB medium. On the day of training, fresh overnight cultures were used to inoculate sterile HIB added to wells in a 96-well plate. An uninfected bacterial control was maintained for all strains used. The initial input cocktail consisted of a 1:1:1 mix of phages AFR43, KEN1, and KEN10 at a total concentration of 1×1010 PFU/mL. Phage lysate from propagated purified phages (for Round 0) and filtered lysate from the previous round of phage training (for all subsequent rounds) was titrated down the columns of the 96-well plate.

The assembled plate was incubated overnight, shaking at 37° C. The following morning, the OD at 600 nm was visualized in a microplate reader. In Panel P1, wells that showed reduction in OD relative to the uninfected control were determined and lysates collected. In Panel P2, only the two most dilute wells showing reduction were collected to select for the best performing possible recombinant/mutant phages. If no reduction in OD was observed, the two least dilute wells were collected. For both panels, all collected lysates were pooled together, filter sterilized through a 0.22 um filter, and stored at 4° C. for subsequent use. To assess for activity on phage resistant strains, pooled lysates were titrated in SM buffer to 10\hat{ }₇ and plated on the strains used in the phage training. Plaque formation was assessed the following morning and plaques collected for follow up analysis.

### Purification of Collected Trained Phage Clones:

Phage clones isolated on previously resistant strains were collected via picking plaques and transferring to 500 μL of sterile SM buffer. 50 uL chloroform was added to assist in destroying any remaining cells and releasing phage still inside. After allowing for equilibration, the plaque suspensions were centrifuged at 5000 g for 5 minutes. The aqueous phase was collected and filter sterilized. This process of picking plaques and filter sterilization was repeated three times and plaque purity was then assessed by confirming uniform plaque morphology.

### Host Range Determination:

Host range is determined by assessing lysis against a 100-strain highly diverse panel of bacterial clinical isolates. Overnight cultures of bacterial strains were pre-pared in HIB medium. On the day of testing, phage stocks of the tested phages are ti-trated to 10\hat{ }−7 in SM buffer. Phage dilution series are spotted on each strain of the di-versity panel. The following day, quality of lysis is assessed. Lysis is scored on a quali-tative system with scores from 0-4. Zero indicates no lysis, one indicates lysis without replication (no plaque formation), 2-4 represent varying levels of lysis with plaque formation. Scores are tallied for a phage against all 100 strains.

### Stability of Host Range Expansion Assessment:

Stability of observed changes in lytic spectra were assessed by conducting serial propagations on a single, phage susceptible bacterial strain. Propagations were con-ducted This is followed by re-isolation and purification of subclones and re-assessment of host range.

### Lysis Dynamics Analysis in 96-Well Plate Format:

Phage lysis and kinetics of lysis are assessed by real time monitoring of clearance of optical density in a 96-well plate format. Outer wells are filled with 100 uL SM buff-er to aid in preventing evaporation during incubation. HIB is inoculated in the morn-ing with overnight culture to reach an OD of 0.05-0.1 and allowed to grow to mid-log (OD600=0.5), shaking at 37° C. Once OD is reached, the culture is placed on ice to min-imize further growth. 100 μL of sterile HIB media is added to one row to serve as a blank. 100 μL of mid-log culture is added to the next row to serve as a phage-free growth control. Finally, the remaining four rows have 95 μL of mid-log culture with 5 μL of phage to reach MOI values desired (typically 10, 1, 0.1, 0.01). The plate is sealed with sterile, clear, gas-permeable plate tape and then placed in a microplate reader (SpectraMax 384, Molecular Devices). The reader is set to 37° C. and is set to read wavelength at 600 nm with reads every 10 minutes for a period of 8 hours or 24 hours. Results of lysis are compared to the uninfected bacterial culture to assess lytic capability of a phage against a particular bacterial strain.

### Endotoxin Purification of Phage Stocks:

Phage stocks intended for use in therapeutic cocktails and in animal experiments were purified of endotoxin. A large volume culture (250 mL) of appropriate bacterial host strain was used to propagate phage to a high titer (>1011 PFU/mL). The phage lysate was pelleted overnight spinning at 6000 g. Lysate was removed, and the pellet re-suspended in 7 mL of SM buffer. This concentrated stock was then further cleaned with octanol washes in triplicate. The final collection was then mixed with 5 mL of p=1.7 CsCl solution and pelleted overnight at 3900 rpm, although initially intended as 4700 g. The speed was lower due to rotor constraints, but fractions were properly formed. Resulting fractions were collected and phage titer was assessed for each fraction. If sufficient phage was in the desired phage fraction, the sample was dialyzed in 1×PBS with 5% MgCl2 over three rounds, switching buffer between each round. The phage sample was dialyzed in a 3 mL 20K cutoff dialysis cassette. Finally dialyzed phage samples were plated again to determine titer and then endotoxin levels were assessed via using the LAL test system, EndoSafe PTS (Charles River) following a modified version of the manufacturer's protocol. In brief, dialyzed phage was diluted in SM buffer. A 1:1 mix of diluted phage and B-G-blocker (Lonza) was prepared and then assessed per manufacturer's protocol.

### Phage Cocktail Preparation for Animal Experiments:

Purified phage stocks were titrated and plated within a week of the start of an animal experiment to confirm phage stock titer. Calculations to generate a phage cocktail are made to ensure that phages reach desired concentration for the experiment without allowing endotoxin levels to reach above FDA recommended limits of 500 EU/kg/hr. Phage cocktails are prepared in 1×-PBS buffer (PBS+10 mM MgSO4). Cocktail doses were prepared the day of dose administration.

### Phage Treatment of Dorsal Wound Model of Infection:

Full thickness dorsal wounds were created in four groups of eight mice each. The wounds were infected with P. aeruginosa PAO1::lux at a dose of 107 CFU. The four groups consisted of a control group, a phage cocktail treated group, an antibiotic treated group, and a phage/antibiotic combination treated group. Treatment regimens were begun four hours post infection and continued for four days. Phage cocktail was administered both topically (TOP) and by intraperitoneal (IP) injection at a dose of 109 PFU made up with equal parts of phages EPa15, EPa18, EPa22, EPa43 and 20176-4-2 in volumes of 200 uL (IP) and 25 uL (TOP). Ceftazidime was used at a dose of 410 mg/kg and administered via IP injection four hours after injection and twice daily following. The antibiotic/phage combination treatment regimen consisted of phage applied topically and ceftazidime delivered via IP injection. Control mice were treated with saline (company). Mice were monitored over the course of the experiment with weight checks (daily), Aranz measurements (taken on Days 0, 3, 6, 10, 15, and 20), and IVIS measurements (taken on Days 1, 2, 3, and 6). Mice were euthanized following closure of wounds.

### Library Preparation and Sequencing

Sequencing libraries was constructed using the KAPA HyperPlus Kit (Roche Diagnostics, Indianapolis, IN) as described previously, [22] with modifications. Briefly, input DNA was tagmented and amplified via PCR. The library was sequenced on an Illumina MiSeq (Illumina, San Diego, CA) with a 600 cycle MiSeq Reagent Kit v3 that produced 300-bp paired end reads.

### Genome Assembly

Raw reads were quality assessed using FastQC 0.11.9, [23] and trimmed with Trimmomatic v0.39, with the following parameters: ILLUMINACLIP, TruSeq3-PE-2.fa:2:30:10; LEADING, 3; TRAILING, 3; SLIDINGWINDOW, 4:24; and MINLEN, 60. Reads were further trimmed with Trimmomatic using the HEADCROP:9 parameter. The phage genome was de novo assembled from the trimmed reads using the Unicycler assembly pipeline

### Genome Annotation

The termini of each assembled phage genome was determined using PhageTerm, Genomes were annotated using Pharokka. Pharokka integrates the gene caller, Phanotate, with evidence from several sources including BLAST searches against cus-tom phage/prophage databases, searches against phage protein domains.

### Genome Variation Analysis

Structural variations, single nucleotide polymorphisms (SNPs), and indels were identi-fied between phage mutant genomes and their respective parent strains using nucdiff.Nucdiff aligns input genomes and detects variants including substitutions, inser-tions, deletions, inversions, and translocations. The R package gggenome, was used to visualize genomic variations. Aligned genomes were provided as input to gen-erate figures depicting SNPs, indels, and structural rearrangements. Default gggenome plotting parameters were used, except for varying figure size and color schemes.

1.2. K. pneumoniae Training

### Bacterial Strains and Bacteriophages Used:

All bacterial strains used in this study are taken from a 100-strain highly diverse panel of bacterial clinical isolates prepared by the MRSN. All bacterial strains were grown in Heart Infusion Broth (HIB) at 37° C., shaking. Cultures were prepared fresh before the start of each experiment, with fresh colonies inoculated in HIB and grown the night before experiments. Strains were selected based on their phage resistance, with strains that are both antibiotic resistant and phage resistant being selected. A susceptible strain, MRSN414780, was used for general propagation of training lysates and to maintain phage titers during training. All phages used in the training were propagated on host bacterial strains. In brief, concentrated phage stock was added to bacterial culture at mid-log phase in propagation media (HIB+glucose, MgCl2, and CaCl2)) and allowed to incubate with the bacterial culture for 4-6 hours or until the culture was cleared, whichever came first. Cultures were then pelleted and phage lysate was filter sterilized by passing through a 0.22 um syringe filter. Filtered phage lysates were used as the initial inoculum for the phage training. Phage lysates were stored, protected from light, at 4° C. Training phages were selected based on their host range coverage and genetic similarity which is important to help facilitate recombination, which is likely a major driver of change in training experiments. The phages all belong to the Jiaodavirus genus which has been reported as having a broad host range, quick lysis time, and efficacy at lower MOIs.

### Appelmans Training:

Appelmans training was conducted with minor modifications as described by Burrowes et al, [Burrowes B H, Molineux I J, Fralick J A. Directed in Vitro Evolution of Therapeutic Bacteriophages: The Appelmans Protocol. Viruses. 2019; 11(3):241. doi.org/10.3390/v11030241]. In brief, phage resistant bacterial clinical isolates were selected. They were grown overnight in HIB medium. On the day of training, fresh overnight cultures were used to inoculate sterile HIB added to wells in a 96-well plate. An uninfected bacterial control was maintained for all strains used. The initial phage inoculum consisted of a 1:1:1:1 mix of KEN22, KEN25, KEN37, and KEN39 to a final concentration of 1×1010 PFU/mL. Phage lysate from propagated purified phages (for Round 0) and filtered lysate from the previous round of phage training (for all subsequent rounds) was titrated down the columns of the 96-well plate.

The assembled plate was then allowed to incubate overnight, shaking at 37° C. The following morning, the OD at 600 nm was visualized in a microplate reader. Wells that showed reduction in OD relative to the uninfected control were tracked. The two most dilute wells showing reduction were collected in order to select for the best performing possible recombinant/mutant phages. If no reduction in OD was observed, the two least dilute wells were collected. All collected lysates were pooled together, filter sterilized through a 0.22 um filter, and stored at 4° C. for subsequent use.

To assess for activity on phage resistant strains, pooled lysates were titrated in SM buffer to 10\hat{ }−7 and plated on the strains used in the phage training. Plaque formation was assessed the following morning and plaques collected for follow up analysis.

### Purification of Collected Trained Phage Clones:

Phage clones isolated on previously resistant strains were collected via picking plaques and transferring to 500 μL of sterile SM buffer. 50 uL chloroform was added to assist in destroying any remaining cells and releasing phage still inside. After allowing for equilibration, the plaque suspensions were centrifuged at 5000 g for 5 minutes. The aqueous phase was collected and filter sterilized. This process of picking plaques and filter sterilization was repeated three times and plaque purity was then assessed by confirming uniform plaque morphology.

### Host Range Determination:

Host range is determined by assessing lysis against a 100-strain highly diverse panel of bacterial clinical isolates. Briefly, overnight cultures of bacterial strains are prepared in HIB medium. On the day of testing, phage stocks of the tested phages are titrated to 10\hat{ }−7 in SM buffer. Phage dilution series are spotted on each strain of the diversity panel. The following day, quality of lysis is assessed. Lysis is scored on a qualitative system with scores from 0-4. Zero indicates no lysis, one indicates lysis without replication (no plaque formation), 2-4 represent varying levels of lysis with plaque formation. Scores are tallied for a phage against all 100 strains.

### Stability of Host Range Expansion Assessment:

Stability of observed changes in lytic spectra were assessed by conducting serial propagations on a single, phage susceptible bacterial strain. This is followed by re-isolation and purification of clones and re-assessment of host range. Host range is compared with the originally isolated trained phage clones.

### Library Preparation and Sequencing

Sequencing libraries was constructed using the KAPA HyperPlus Kit (Roche Diagnostics, Indianapolis, IN) as described previously [29] with modifications. Briefly, input DNA was tagmented and amplified via PCR. The library was sequenced on an Illumina MiSeq (Illumina, San Diego, CA) with a 600 cycle MiSeq Reagent Kit v3 that produced 300-bp paired end reads.

### Genome Assembly

Raw reads were quality assessed using FastQC 0.11.9 and trimmed with Trimmomatic v0.39, with the following parameters: ILLUMINACLIP, TruSeq3-PE-2.fa:2:30:10; LEADING, 3; TRAILING, 3; SLIDINGWINDOW, 4:24; and MINLEN, 60. Reads were further trimmed with Trimmomatic using the HEADCROP:9 parameter. The phage genome was de novo assembled from the trimmed reads using the Unicycler assembly pipeline.

### Genome Annotation

The termini of each assembled phage genome was determined using PhageTerm. Genomes were annotated using Pharokka. Pharokka integrates the gene caller, Phanotate, with evidence from several sources including BLAST searches against custom phage/prophage databases, searches against phage protein domains.

### Genome Variation Analysis

Structural variations, single nucleotide polymorphisms (SNPs), and indels were identified between phage mutant genomes and their respective parent strains using nucdiff. Nucdiff aligns input genomes and detects variants including substitutions, insertions, deletions, inversions, and translocations. The R package gggenome was used to visualize genomic variations. Aligned genomes were provided as input to generate figures depicting SNPs, indels, and structural rearrangements. Default genome plotting parameters were used, except for varying figure size and color schemes.

### Example 2. Phage Training Leads to Phage Clones with Altered and Expanded Host Ranges

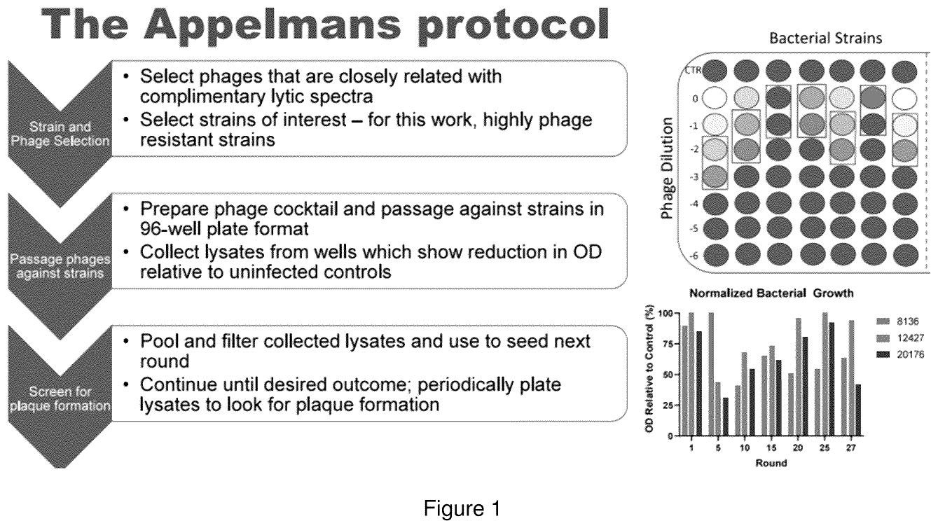

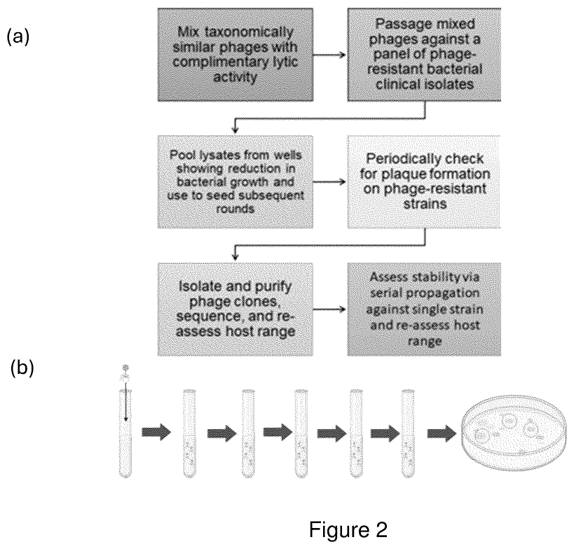

The protocol is schematically summarized in FIG. 1 and FIG. 2. Phages were combined at an equal ratio of each phage titer to yield a phage cocktail of 1×10¹⁰ pfu/mL. Using a 96-well microtiter plate, 100 μL of serial 10-fold dilutions (10⁰ to 10⁻⁶) of the phage cocktail were added to 100 μL of double-strength LB containing 1 μL of an overnight bacterial culture of a phage-susceptible strain. 10-12 bacterial strains can be tested on each plate. For each bacterial strain, the top well was used for a control, i.e., bacterial strain without phage addition. After overnight incubation at 37° C. on a shaking platform at 200 rpm, the plates were visually inspected. Wells showing reduced bacterial growth and complete lysis were pooled. Pooled lysates were cleared by vortex mixing with 1:100 CHCl₃, followed by centrifugation (15,000×g for 15 min). The lysate was termed the round 1 cocktail and was used to initiate the next round of the protocol using the same set of bacterial strains.

In the experiments described here, genetically similar phages with complementary activity, were mixed. Three Pseudomonas phages, which are members of the Phikmvvirus genus (KEN1, KEN10, AFR43), were selected for the Pseudomonas phage training, and four Klebsiella phages, which are members of the Jiaodavirus genus (KEN22, KEN25, KEN37, KEN39), for the Klebsiella phage training (Table 1). This was to facilitate DNA recombination events in phages active against different strains of the same bacterial species so that their progeny would gain the ability to lyse additional bacterial strains.

Pseudomonas phage cocktail was passaged serially against ‘phage training’ panels including 10-12 phage-resistant P. aeruginosa strains and one phage permissive strain (e.g., PAO1) (Table 2a). Klebsiella phages cocktail was passaged serially against a panel including 10 phage-resistant bacterial strains and one phage permissive strain (MRSN414780) (Table 2b). Then, phage lysates were collected from the wells showing reduced bacterial growth, pooled, and passaged against the same panel of bacterial strains over successive rounds.

Three sets of training panels were used in this work (Table 3). All phage variants isolated from panel Pa-OK showed altered specificity, reduction in activity and developed as specialists. Several phage clones from panels Pa-KB and Kp-KB displayed expanded host range and were used in further work.

TABLE 1
Characteristics of bacteriophages selected for training

| | Genome, size | | | Host | Training |
|---|---|---|---|---|---|
| Phage ID | bp | Family | Genus | range, % | Panels |
| AFR43 | 43,008 | Autographiviridae | PhilKMVvirus | 37 | Pseudomonas |
| KEN1 | 42,986 | Autographiviridae | PhilKMVvirus | 40 | Pseudomonas |
| KEN10 | 42,678 | Autographiviridae | PhilKMVvirus | 37 | Pseudomonas |
| KEN22 | 167,213 | Straboviridae | Jiaodavirus | 26 | Klebsiella |
| KEN26 | 165,574 | Straboviridae | Jiaodavirus | 31 | Klebsiella |
| KEN37 | 166,503 | Straboviridae | Jiaodavirus | 34 | Klebsiella |
| KEN39 | 166,254 | Straboviridae | Jiaodavirus | 36 | Klebsiella |

TABLE 2
Characteristics of the bacterial strains
selected for phage training panels

| | | | |
|---|---|---|---|
| Strain ID | Antibiotic resistance | Phage susceptibility | Panel |

(a) Pseudomonas phage training panel strains
(a) Pseudomonas phage training panel strains

| | | | |
|---|---|---|---|
| MRSN317 | MDR | XPR | KB/OK |
| MRSN552 | susceptible | MPR | KB/OK |
| MRSN1388 | Susceptible | XPR | KB |
| MRSN2144 | Susceptible | MPR | OK |
| MRSN6220 | PDR | PPR | KB |
| MRSN6678 | XDR | PPR | KB |
| MRSN8130 | MDR | MPR | OK |
| MRSN8136 | XDR | XPR | KB |
| MRSN8914 | XDR | XPR | OK |
| MRSN12427 | ND | PPR | KB |
| MRSN13488 | Susceptible | PPR | KB/OK |
| MRSN16344 | XDR | MPR | OK |
| MRSN20176 | XDR | XPR | KB/OK |
| MRSN20193 | ND | XPR | KB |
| MRSN25678 | MDR | PPR | KB |
| MRSN26263 | Susceptible | PPR | KB |
| MRSN358800 | XDR | XPR | OK |
| PAO1 | Susceptible | Susceptible | KB/OK |
| MRSN351791 | MDR | MPR | |

(b) Klebsiella phage training panel strains

| | | | |
|---|---|---|---|
| MRSN4759 | MDR | XPR | KB |
| MRSN6778 | MDR | PPR | KB |
| MRSN15687 | MDR | XPR | KB |
| MRSN15882 | MDR | PPR | KB |
| MRSN22232 | XDR | PPR | KB |
| MRSN27989 | MDR | PPR | KB |
| MRSN479404 | XDR | PPR | KB |
| MRSN511348 | XDR | XPR | KB |
| MRSN614201 | MDR | PPR | KB |
| MRSN681054 | XDR | PPR | KB |
| MRSN414780 | XDR | Susceptible | KB |

PDR/PPR (pan-drug/phage resistant): no tested drugs/phages kill;
XDR/XPR (extensively drug/phage resistant): fewer than 3 drugs/phages kill;
MDR/MPR (multi-drug/phage resistant): resistant to greater than third of tested drugs/phages;
ND: no data

TABLE 3
Host Range coverage of collected trained phages

| Phage ID | Host Strain | Training Panel | Host Range |
|---|---|---|---|
| 20176-1 | P. aeruginosa 20176 | KB-Pa | 27% |
| 20176-2 | P. aeruginosa 20176 | KB-Pa | 29% |
| 20176-3 | P. aeruginosa 20176 | KB-Pa | 40% |
| 20176-4 | P. aeruginosa 20176 | KB-Pa | 42% |
| 20176-5 | P. aeruginosa 20176 | KB-Pa | 43% |
| 20176-6 | P. aeruginosa 20176 | KB-Pa | 42% |
| 20176-7 | P. aeruginosa 20176 | KB-Pa | 43% |
| 20176-8 | P. aeruginosa 20176 | KB-Pa | 33% |
| 20176-9 | P. aeruginosa 20176 | KB-Pa | 42% |
| 20176-10 | P. aeruginosa 20176 | KB-Pa | 42% |
| 15882-1 | K. pneumoniae 15882 | KB-Kp | 33% |
| 15882-3 | K. pneumoniae 15882 | KB-Kp | 44% |
| 15882-5 | K. pneumoniae 15882 | KB-Kp | 36% |
| 15882-6 | K. pneumoniae 15882 | KB-Kp | 40% |
| 15882-7 | K. pneumoniae 15882 | KB-Kp | 38% |
| 15882-8 | K. pneumoniae 15882 | KB-Kp | 14% |
| 15882-4 | K. pneumoniae 27989 | KB-Kp | 11% |
| 15882-11 | K. pneumoniae 27989 | KB-Kp | 9% |
| 15882-12 | K. pneumoniae 27989 | KB-Kp | 10% |
| 552-2 | P. aeruginosa 552 | OK-Pa | 3% |
| 552-5 | P. aeruginosa 552 | OK-Pa | 3% |
| 2144-1.2 | P. aeruginosa 2144 | OK-Pa | 8% |
| 2144-5.1 | P. aeruginosa 2144 | OK-Pa | 8% |
| 2144-5.2 | P. aeruginosa 2144 | OK-Pa | 7% |
| 2144-6.2 | P. aeruginosa 2144 | OK-Pa | 8% |
| 2144-7.2 | P. aeruginosa 2144 | OK-Pa | 8% |
| 2144-8.1 | P. aeruginosa 2144 | OK-Pa | 8% |
| 8130-3.1 | P. aeruginosa 8130 | OK-Pa | 4% |
| 8130-3.2 | P. aeruginosa 8130 | OK-Pa | 4% |
| 8130-4.1 | P. aeruginosa 8130 | OK-Pa | 5% |
| 8130-5.1 | P. aeruginosa 8130 | OK-Pa | 5% |
| 8130-5.2 | P. aeruginosa 8130 | OK-Pa | 5% |
| 8914-1 | P. aeruginosa 8914 | OK-Pa | 10% |
| 8914-3 | P. aeruginosa 8914 | OK-Pa | 10% |
| 8914-4 | P. aeruginosa 8914 | OK-Pa | 10% |
| 8914-5 | P. aeruginosa 8914 | OK-Pa | 10% |
| 8914-7 | P. aeruginosa 8914 | OK-Pa | 10% |
| 8914-8 | P. aeruginosa 8914 | OK-Pa | 10% |
| 16344-A1/1 | P. aeruginosa 16344 | OK-Pa | 4% |
| 16344-A1/2 | P. aeruginosa 16344 | OK-Pa | 4% |
| 16344-A2/1 | P. aeruginosa 16344 | OK-Pa | 4% |
| 16344-A2/2 | P. aeruginosa 16344 | OK-Pa | 4% |
| 16344-1./2 | P. aeruginosa 16344 | OK-Pa | 4% |
| 16344-5./2 | P. aeruginosa 16344 | OK-Pa | 4% |
| 20176-35-2 | P. aeruginosa 20176 | OK-Pa | 2% |
| 358800-H1 | P. aeruginosa 358800 | OK-Pa | 38% |
| 358800-H2 | P. aeruginosa 358800 | OK-Pa | 38% |
| 358800-H3 | P. aeruginosa 358800 | OK-Pa | 38% |
| 358800-H6 | P. aeruginosa 358800 | OK-Pa | 37% |

2.1. P. aeruginosa Phage Training

Phage training was conducted per the modified Appelmans training and continued until phage clones with desired attributes (ability to form plaques on phage resistant strains) were collected. Training was conducted over two independent experiments which employed different strains but the same cocktail of phages. Panel 1 phage training occurred prior to the start of the Panel 2 training and information obtained from this run was used to improve set-up for the P2 training. Lysis was monitored each round and strains showing lysis were periodically screened to look for plaque formation.

After 49 rounds of training in P1 panel, the pooled lysate collected from the final round was able to lyse 50% of the diversity panel. Plaques were collected on numerous strains and phages were isolated on MRSN552, MRSN2144, MRSN8130, MRSN8914, MRSN16344, MRSN20176, and MRSN358800 and successfully purified. Most of the purified phages collected from this panel had a narrow host range, ranging from 2% to 38% at most. Despite loss in overall activity, there were still differences in host range from the parental phages; these phages displayed altered lytic spectra, lysing strains that the parental phages could not, including highly phage resistant bacterial strains. Phages isolated on MRSN358800 had the best performance with four phages (H1, H2, H3, H4) isolated on that strain lysing 38%, 38%, 38% and 37% respectively. This puts these phages as relatively equivalent in host range, but with an altered lytic spectrum. Phages isolated on all other strains lysed 10% or fewer strains of the 100-strain diversity panel.

Clones collected from the P1 training panel developed at different rounds. Plaques were first observed on MRSN8130 and MRSN351791 following round 1. Plaques were first observed on MRSN2144, MRSN16344, MRSN8914, MRSN20176, and MRSN552 at rounds 4, 9, 18, 35, and 44 respectively. Plaques collected on MRSN358800 were collected by passaging lysate from MRSN20176 on MRSN358800. In some cases, plaques were lost and then reappeared at later rounds of training. In cases where collected phages were isolated earlier, they were more often narrow host range. For example, phages collected on MRSN8130, which had plaques appear after only one round of training had host ranges of Following 20 rounds of training in the P2 panel, ten plaques were isolated on a formerly phage-resistant bacterial isolate, MRSN20176. Plaques were observed following round five of training but were incredibly faint. This raised two concerns: that these phages would be difficult to propagate or handle in the laboratory, and that not enough time had passed for expansion in host range. So, training was continued for further rounds until plaques were finally collected on round 20. These plaques were more robust but unable to fully clear the bacteria from the plaque. After purification of the collected phage clones, they were assessed for host range against a 100-strain highly diverse panel of Pseudomonas aeruginosa clinical isolates. Additionally, the three parental phages used in the training were also plated against the diversity panel. The parental phages had host ranges of 37-40%, consisting of all strains lysed with productive plaque formation. Each parental phage also displayed non-replicative lysis on a small additional number of strains.

Following assessment of the trained clones, it was found that six of ten collected iso-lates displayed broader host range than the best performing parental phage. These clones had host ranges of 42-44% lysis. Additionally, all clones displayed non-replicative lysis on additional strains.

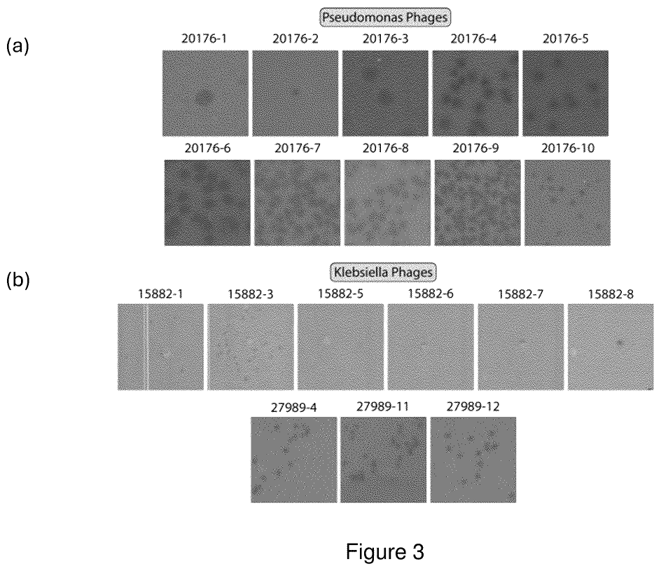

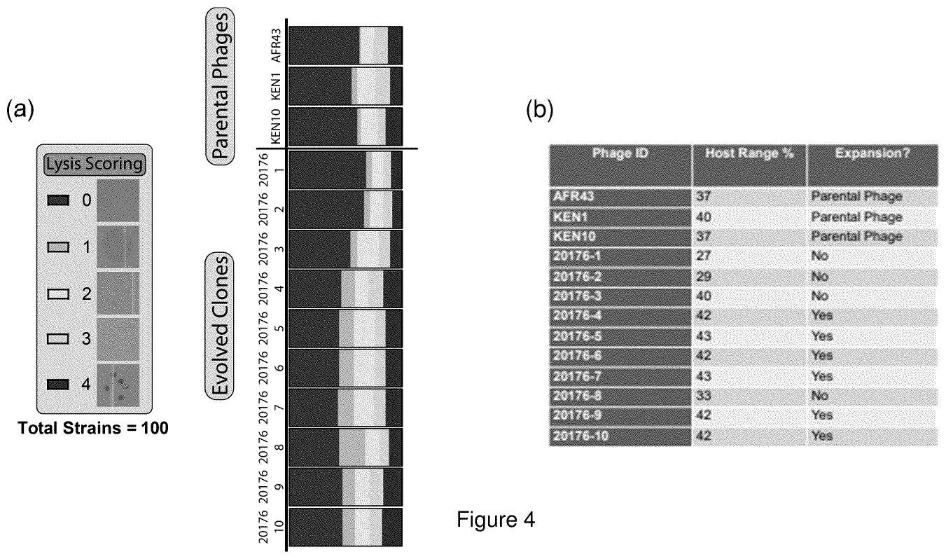

FIG. 3a shows plaque morphology of collected phage clones from KB-Pa phage training panel. Plaques are on agar plates with lawns of MRSN20176 grown on HIB plates with a 0.7% agar HIB top layer. FIG. 4 summarizes the results of host range of (a) trained phages collected from panels P1 and (b) P2 final round (49) lysate from plating results on 100 strains of P. aeruginosa.

2.2. K. pneumoniae Phage Training Leads to Phage Clones with Altered Lytic Spectra.

Following 10 rounds of phage training, multiple phage clones infecting highly phage resistant strains were isolated, which were previously resistant to the parental phages. Phages clones were collected from two highly resistant strains, MRSN15882 and MRSN27989. Phage clones collected on these strains were weakly lytic, with small, faint plaques, (FIG. 3). Ultimately, three clones were isolated and purified from MRSN27989, and six clones were isolated and purified from MRSN15882. Additional plaques were originally identified on other resistant strains, including additional plaques on MRSN15882 and MRSN27989, and plaques on MRSN15687 and MRSN681054, but were seemingly unstable and lost before being purified, or were recalcitrant to propagation reaching a workable phage titer. Following three rounds of plaque isolation, phage clones were plated on 100 strains from a highly diverse panel of clinical isolates.

Full characterization was focused on those phage clones that proved more amenable to handling in the lab. The nine phage clones assessed against the 100-strain diversity panel displayed altered lytic spectra and in three cases, expanded lytic spectra. Additionally, parental phage clones KEN22, KEN25, KEN37, and KEN39 were plated on the panel at the same time. Phage clones 15882-3, 15882-6, and 15882-7 had expanded activity with activity against 44, 40, and 38% of the panel respectively. Compared with the best performing parental phage, KEN39, which had a host range of 36%, this represents an expansion of 8, 4, or 2% over KEN39. Increasing lytic activity against eight strains is a substantial increase in activity in that phage clone. No other phage clones collected from the training project saw expansion in activity. 15882-1 and 15882-5 saw activity roughly equivalent to the parental phages with 33% and 36% activity respectively. Finally, phage clones 15882-8 and 27989-4, 27989-11 and 27989-12 showed dramatic loss in activity with host ranges of 14, 11, 9, and 10% respectively.

For all phage clones, the quality of lysis indicated by assessing plaque quality indicates expansion of weakly lytic plaques. Plaques were largely turbid, with no full clearance of the bacterial culture. While parental phages only robustly lysed strains with full clearance, all trained clones only lysed a single strain with 4+ activity. While activity was expanded, the quality of action was reduced. Most expansion in activity was of a faint, weak plaque. Additionally, the trained phages acquired from MRSN15882, with the exception of 15882-8, gained further activity with respect to non-replicative lysis.

Even for phages with reduced or equivalent host range, the strains lysed by these trained clones differs from the parental phages. First and foremost, these phage clones are capable of lysing highly phage resistant strains (MRSN15882 and MRSN27989).

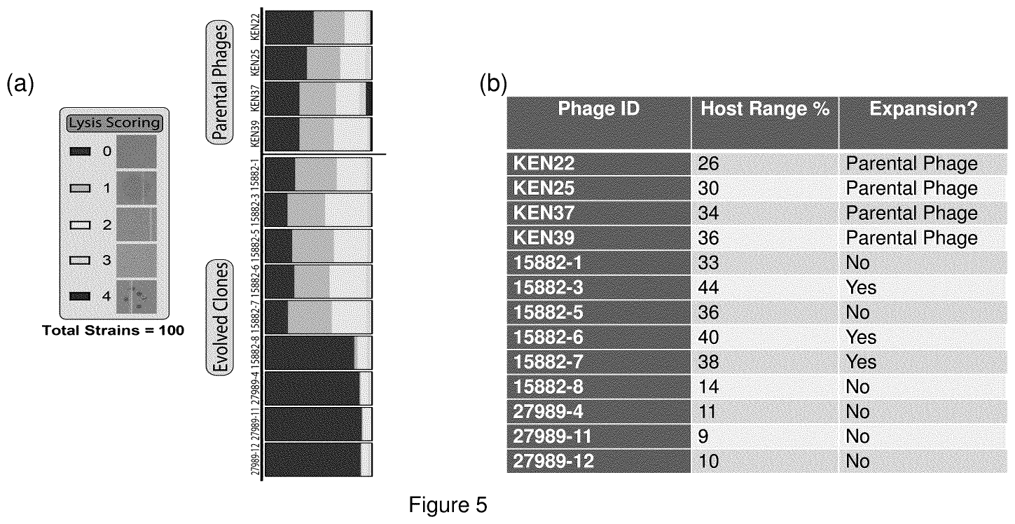

FIG. 3b shows plaque morphology of phage clones collected following 10 rounds of training on phage-resistant strains MRSN15882 and MRSN27989. FIG. 5 shows the result of host ranges of trained phages and parental phages on 100-strain diversity panel strains following 10 rounds of training. Lysis scoring is derived from a qualitative scoring system based on plaque formation and quality of observed plaques.

### Example 3. Phage Host Range Alteration is Largely Stable Even after Training is Completed

3.1. P. aeruginosa Phage Host Range Alteration is Largely Stable Even after Training is Complete.

The stability of host range expansion was assessed via taking purified phages and serially propagating them against strain PAO1. Clones that showed expansion, except for 20176-9 and 20176-10, which proved difficult to propagate to high titer, were serially propagated for stability assessment. Ultimately, four phages were assessed in this manner, 20176-4, 20176-5, 20176-6, and 20176-7. Three subclones were isolated following serial propagations. The host range of these subclones was re-assessed following purification. Ultimately, most phage clones retained similar activity as the originally isolated trained phage. In several cases, activity was lost but was still higher than the parental phages. This was the case for 20176-4-3. In two cases, host range coverage expanded relative to the trained phage clone. 20176-4-1 and 20176-4-2 both displayed increased host range coverage with 44% and 45% coverage when tested against the 100-strain diversity panel. Non-replicative lysis was observed on an expanded number of strains following stability assessment for all stability phage clones relative to the originally trained phages and the parental phages.

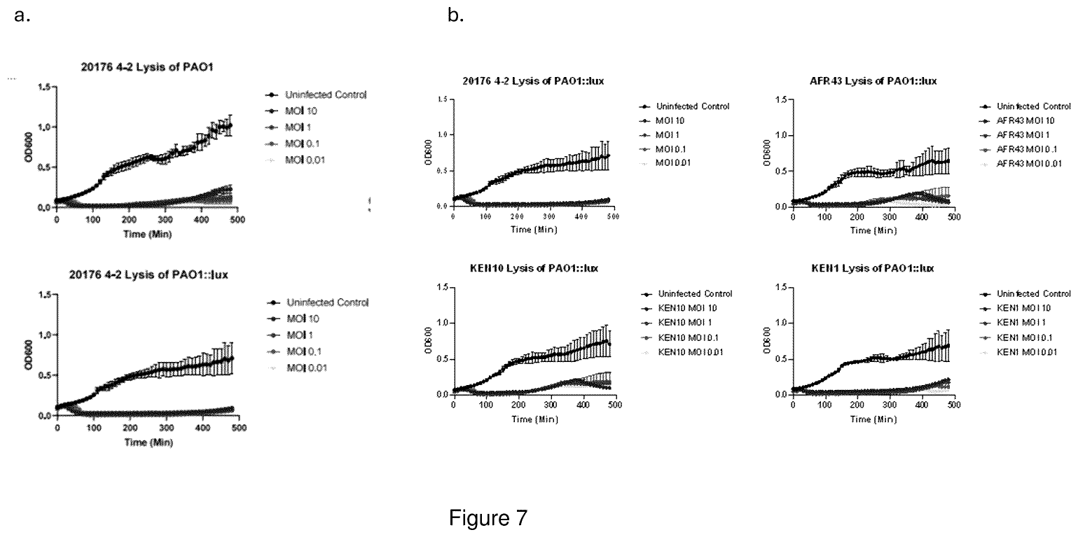

FIG. 7 summarizes host range expansion assessment. Three subclones were plated against 100 strains of P. aeruginosa following plaque purification and host ranges determined.

3.2. K. pneumoniae Host Range Expansion is not Stable Following Removal of Phages from Training Panel.

Once purified, phage clones were assessed for stability of observed changes to host range by passaging phages against a highly phage susceptible strain serially. Following five serial propagations on phage susceptible strain MRSN414780, phage clones were recollected and host range reassessed. Three subclones were collected from propagated lysate of 15882-3, 15882-6, and 15882-7. Interestingly, the proportion of strains lysing with full clearance of the bacterial lawn (3+ and 4+ activity) increased in all subclones. In the originally trained clones, only 1 strain, MRSN414780, was lysed with 4+ activity. That increased to 4 strains for 15882-3 subclones, 5 strains for 15882-6 subclones, and 4 strains for 15882-7 subclones, (FIG. 3). The strains lysed with higher level activity primarily expressed 03b and O1v1 LPS type loci.

The ultimate goal of our experiment was to isolate phages with expanded host range that could be incorporated in a therapeutic phage cocktail. While phage clones were not stable, improvement in host range demonstrated that the phages had expanded activity. With the incorporation of phage 15882-3 it could theoretically reach 75% coverage. With the best subclone collected following stability testing, we could still reach 67% coverage. In both cases, coverage was improved relative to the best performing phage cocktail in this lab, KPM1, which collectively covers only 71% of the 100-strain diversity panel. This is because the trained phages have a broader host range, and a better coverage against other strains not already targeted.

3.3. Lysis Dynamics of Trained K. pneumoniae Phages Compared with Parental Phages:

A therapeutic phage needs not only to form plaques on strains of interest, it also must be able to lyse the strain in a timely manner and should ideally suppress phage resistant mutants from developing. To assess these attributes of the trained phages, lysis dynamics of the trained phages was examined, which showed expanded coverage and compared with parental phages KEN22, KEN25, KEN37 and KEN39.

FIG. 7 summarizes host range of subclones collected following serial propagation on permissive host MRSN414780. Subclones were compared with originally collected evolved clones to determine host ranges following plating on 100-strain diversity panel.

Overall, following multiple rounds of training, phage clones displaying altered specificity and/or expanded host ranges compared with the parental phages were obtained when tested against genetically diverse 100-strain panels of global diversity panel of MDR P. aeruginosa or K. pneumoniae.

Altogether, 31 P. aeruginosa phages and 9 K. pneumoniae phages with altered host specificity were isolated and characterized. They were individually plated on a previously pan-phage-resistant clinical strain of P. aeruginosa, MRSN 20176 or K. pneumoniae MRSN 15882. Among these phage clones, 4 Pseudomonas phages and 3 Klebsiella phages possessed expanded host ranges. These clones were collected from phage-resistant strains P. aeruginosa MRSN 20176 and K. pneumoniae MRSN 15882.

Parental Pseudomonas phages KEN1, KEN10 and AFR43 each covered 37-40% of diversity panel strains. Pseudomonas phage clones 20176-4, 20176-5, 20176-6, and 20176-7 covered 42%, 42%, 43% and 43% of P. aeruginosa strains, respectively. Parental Klebsiella phages (KEN22, KEN25, KEN37, KEN39) each covered 26-36% of diversity panel strains. Klebsiella phage clones 15882-3, 15882-6, and 15882-7 covered 44%, 40%, and 38%, respectively.

The stability of phages was tested with the altered Pseudomonas phage clones having expanded host ranges to determine whether the observed spectral expansion would be retained after repeated propagation on a single host. The host range expansion was retained by most of the altered Pseudomonas clones. Among them, phage clones 20176-4, 20176-5, 20176-6, and 20176-7 were passaged against phage-permissive strain PAO1 serially over five rounds.

Lysates were then plated, and three clones were collected from each, purified, and re-plated against the 100-strain global diversity set to assess host range. Surprisingly, phage subclones collected from 20176-4 showed an increase in host range, while the remaining clones showed either retention of host range or a reduction. Phage subclone 20176-4-2, one of the subclones of 20176-4, was selected for use in phage cocktail development.

It is unclear why some phages retained activity after serial propagation better than others. K. pneumoniae phages collected using MRSN15882 showed greater instability in general than P. aeruginosa phages isolated using MRSN20176. The increase in higher quality lysis might suggest that given enough time, these K. pneumoniae phages may develop as specialists (phages with high lytic capability against a small subset of strains).

### Example 4. P. aeruginosa Phage Clone 20176-4-2 Shows Therapeutic Potential During Characterization

4.1. Phage 20176-4-2 Robustly Lyses Infection Challenge Strains of P. aeruginosa

Phage clone 20176-4-2 was selected to move forward for characterization to determine if the phage would make for a viable therapeutic phage. The initial work focused on lysis dynamics of this phage when tested against our infection challenge strains. Phage 20176-4-2 was mixed at a range of MOI values from 10 to 0.01 and allowed to incubate and mix with common infection challenge strains PAO1 and PAO1::lux, a modified version of PAO1 encoding a luciferase reporter cassette.

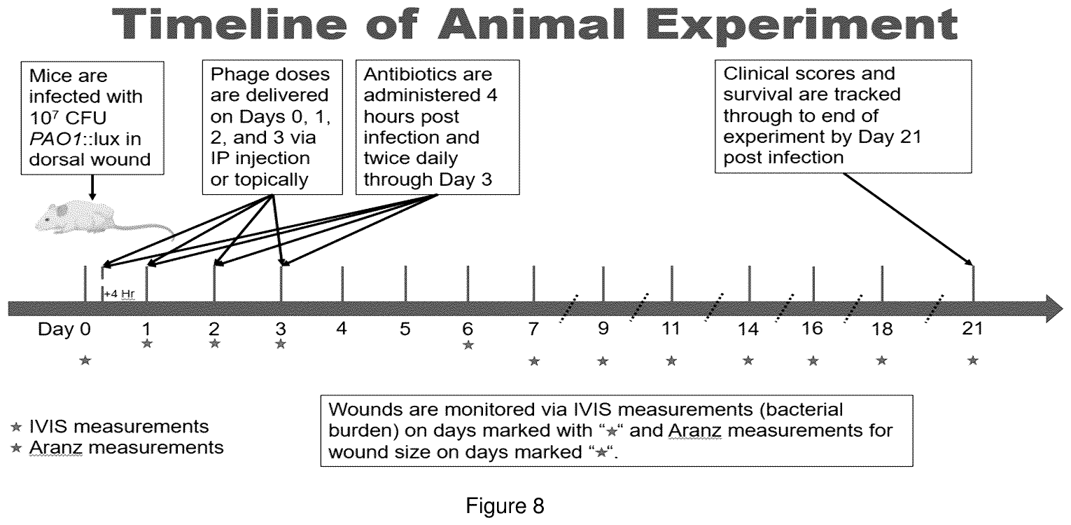

FIG. 8 sows lysis curves of trained phage 20176-4-2 compared with untrained parental phages on infection challenge strain PAO1::lux at MOI ratios of 10-0.01.

Phage 20176-4-2 showed robust lysis against the infection challenge strains, rapidly clearing the culture to the limit of detection at all tested MOI values. Bacterial culture was maintained below the limit of detection for up to 8 hours at which point, out-growth of probable phage resistant mutants took place. However, the rate of out-growth was highly dependent on the concentration of phage added, with outgrowth occurring more frequently in the highest concentrations of phage.

While 20176-4-2 is capable of forming plaques on strain MRSN20176, it does not robustly lyse the strain in liquid culture. At the same range of MOI values as before, the phage is unable to clear the culture to the limit of detection.

When compared with parental phages KEN1, KEN10, and AFR43, lysis of infection challenge strains shows minor differences. The culture is cleared to the limit of detection within similar amount of time, approximately one hour after mixing phage with culture. The infection challenge strain used here is broadly phage susceptible, including to all parental phages and our trained phage. All tested phages can suppress resistant outgrowth for only some time, with the trained phage suppressing for approximately 6 hours compared to 2-3 hours for AFR43 or KEN10, or 4-5 hours for KEN1. The longer suppression of bacterial resistant outgrowth and maintained rapid killing activity demonstrate that this phage has strong potential for therapeutic use.

Next, endotoxin was removed from the phage 20176-4-2 preparation via octanol/cesium chloride fractionation for incorporation into a phage cocktail (endotoxin concentration 0.00912×10⁶ EU/mL and phage titer 7E10\hat{ }11). The phage maintained stable titer over two months of storage (Table 4).

TABLE 4
Titer of phage stock over 2-month period.

| Time point | Phage Titer (PFU/mL |
|---|---|
| 1 week | 5E10\hat{ }9 |
| 1 month | 1.4E10\hat{ }10 |
| 2 months | 2E10\hat{ }9 |

Phage 20176 4-2 was assessed for compatibility and stability by mixing it with other top performing phages from the phage library, and for in vitro killing efficacy (FIG. 8). When the phage at various multiplicity of infection (MOI) was tested for antibacterial effect, phage clone 20176-4-2 robustly killed infection/challenge strains of PAO1 P. aeruginosa strain. For testing the antibacterial effect on a recombinant PAO1 strain, PAO1::lux, with which mice would be infected for in vivo study, the same number of MOI was tested. The phage infection in the recombinant PAO1::lux strain showed very similar antibacterial effect, verifying that lux gene recombination into the bacterial strain did not affect susceptibility of the bacteria.

### Example 5. Phage Sequence Analysis of Trained Phage Clones

The phage clones were sequenced to detect gene recombination, mutations, and single nucleotide polymorphisms (SNP) events that might be driving the observed phenotypic changes. Upon deeper analysis, four major sites of recombination and the presence of numerous SNPs were observed when compared with the most closely similar parental phage genome. Point mutations were identified in genes predicted to encode tail fibers. Additionally, mutations and recombination events were identified in genes predicted to encode DNA polymerase and RNA polymerase and other phage structural proteins (Table 5).

TABLE 5
Observed recombination events in trained phages

| Recombination | Region | Genes in |
|---|---|---|
| Event | Impacted | Region |
| Recombination | 2928-3362 | Two hypothetical genes |
| Region 1 | | Nucleotidyltransferase |
| | | DNA ligase |
| Recombination | 14649-15409 | DNA polymerase, two |
| Region 2 | | additional hypothetical |
| | | genes |
| Recombination | 20202-23559 | RNA polymerase, two |
| Region 3 | | hypothetical proteins virion |
| | | structure protein, head-tail |
| | | adapter |
| Recombination | 33487-33918 | Internal virion protein |
| Region 4 | 34806-36125 | endolysin domain |

Some phage variants gained the ability to lyse previously phage-resistant strains but lost the ability to lyse previously susceptible strains. Whole-genome sequencing results revealed that significant mutations had accumulated in the variants with altered specificity relative to their parental phages. Genomic analysis to identify the mutations responsible for host range alteration or expansion is still in progress.

Host range expansion was observed for six P. aeruginosa phage clones: 20176-4, 20176-5, 20176-6, 20176-7, 20176-9, and 20176-10. Despite possessing divergent host ranges, phage clones isolated from MRSN20176 had identical sequences. Single nucleotide polymorphisms (SNPs) were observed in the 20176 clones relative to the parental phages, including in expected locations like genes predicted to encode tail fibers. The 20176 phage clones were most similar to AFR43.

Host range expansion was only observed in Klebsiella phages isolated from strain MRSN15882, with clones 15882-3, 15882-6, and 15882-7 showing increased coverage. All other Klebsiella evolved clones showed a high loss of activity, (FIG. 3). Phage clones from MRSN15882 had more sequence divergence. Recombination events and SNPs were observed in expected locations such as genes encoding tail fibers.

### Example 6. Therapeutic Effect of PAM3T Treatment in a Mouse Wound Infection Model

Phage 20176-4-2 was incorporated into a modified version of a therapeutic Pseudomonas phage cocktail and tested in a dorsal wound model of infection in mice.

Purified stocks of 20176-4-2 were used to replace phages from an existing Pseudomonas cocktail in development within this lab, WRAIR_PAM3. This cocktail consists of six phages that collectively cover 85% of the diversity panel. Phage 20176-4-2 replaced two phages of the PAM3 cocktail, EPa11 and EPa16, to generate a five-phage cocktail which we termed WRAIR_PAM3T. All phages were purified via a coupled octanol ex-traction and cesium chloride centrifugation. This cocktail collectively covered 85% of the diversity panel, but with one fewer phage.

In summary, using the prepared 20176-4-2 phage, new therapeutic phage cocktail WRAIR_PAM3T was made. The current phage cocktail targeting P. aeruginosa, WRAIR_PAM3, consists of six phages, which collectively target 85 strains of 100 strain global diversity panel. To make a new phage cocktail WRAIR_PAM3T, two phage components of WRAIR_PAM3 were removed, and the “trained” phage 20176 4-2 was added (Table 6). Thus, PAM3T consists of only five phages but has expanded activity, with a combined host range of 85% of the diversity panel.

TABLE 6
Improved phage cocktail PAM3T against P. aeruginosa

| | | | | | | | | Host | | PAM |
|---|---|---|---|---|---|---|---|---|---|---|
| | | | | Host | Host | | | range | Comple- | ho |
| Phage mix | Component | Family | Genus | receptor | resistance | Antagonism | Antibiofilm | (% | mentarity | range |
| PAM 3 | EPa11 | Former Myoviridae | Pbunavirus | L-OSA, | Medium | — | −/++ | 51 | High | 83% |
| 6 components | | | | VL-OSA | | | | | | |
| | EPa15 | Former Myoviridae | Pbunavirus | L-OSA, | NT | — | −/++ | 54 | High | |
| | | | | VL-OSA | | | | | | |
| | EPa16 | Former Myoviridae | Nankokuvirus | A, B | Medium | — | −/++ | 35 | High | |
| | | | | bands | | | | | | |
| | EPa18 | Former Myoviridae | Nankokuvirus | A, B | NT | — | +/++ | 38 | High | |
| | | | | bands | | | | | | |
| | EPa22 | Former Myoviridae | Pbunavirus | L-OSA, | Medium | — | −/++ | 52 | High | |
| | | | | VL-OSA | | | | | | |
| | EPa43 | Mesyanzinovvirdae | Abidjanvirus | LPS | Low | — | −/+ | 37 | High | |
| | | | | core | | | | | | |
| PAM3T | EPa15 | Former Myoviridae | Pbunavirus | L-OSA, | NT | — | −/++ | 54 | High | 85% |
| 5 components | | | | VL-OSA | | | | | | |
| | EPa18 | Former Myoviridae | Nankokuvirus | A, B | NT | — | +/++ | 38 | High | |
| | | | | bands | | | | | | |
| | EPa22 | Former Myoviridae | Pbunavirus | L-OSA, | Medium | — | −/++ | 52 | High | |
| | | | | VL-OSA | | | | | | |
| | EPa43 | Mesyanzinovviridae | Abidjanvirus | LPS | Low | — | −/+ | 37 | High | |
| | | | | core | | | | | | |
| | 20176-4-2 | Autographiviridae | Phikmvvirus | NT | NT | — | NT | 46 | High | |

PAM3T was then tested in a mouse dorsal wound model of infection with P. aeruginosa strain PAO1::lux, with four phage doses provided daily both topically and intraperitoneally (Table 7 and FIG. 7). The therapeutic effect of phage cocktail PAM3T (Group 3) was compared with ceftazidime antibiotic treatment (Group 2), which reduced bacterial luminescence in wounds. However, the most rapid and robust antibacterial effect was observed for the PMT3T-ceftazidime antibiotic combo-treated group (Group 4).

To assess the efficacy of the modified PAM3T cocktail, a mouse dorsal wound infection model was used. Mice were infected with 10\hat{ }7 CFU of strain P. aeruginosa PAO1::lux, which encodes a luciferase to allow for monitoring bacterial burden in the wound by bioluminescence. Mice were treated with four groups: a saline control, ceftazidime alone, PAM3T alone, and a combinatorial treatment of PAM3T and ceftazidime. Wound size, clinical scores, and mortality were tracked over a period of 21 days. Saline treated mice displayed a high mortality rate during the experiment, with 7 of 8 mice dead by 3 days post infection. One mouse from the ceftazidime alone group died by Day 5. All mice treated with PAM3T or with the PAM3T and ceftazidime com-bination survived the full course of the experiment.

Wound size measurements showed that wounds were larger in mice treated with only saline or ceftazidime alone. Ceftazidime treated mice had average wound perimeters increase to over 29.29 mm by Day 7. On the other hand, phage treated mice showed maximal wound sizes reaching 22.5 mm with phage treatment alone and 17.5 mm with phage/antibiotic combination treatment, (Fig #). Wound closure occurred faster in cocktail treated mice. The first mice to show wound closure occurred on Day 14 and occurred only in PAM3T and PAM3T+ceftazidime treated mice. The mean time to wound closure in PAM3T and PAM3T+ceftazidime treated mice was 16.8 and 17.0 days respectively compared with 19.9 days for antibiotic treated mice.

The bacterial burden in the wounds of mice treated with the phage cocktail was dramatically reduced as detected by IVIS measurements. By Day 3, PAM3T alone treated mice showed modest reduction in bacterial burden compared to ceftazidime alone. However, PAM3T+ceftazidime treated mice had nearly undetectable levels of bacteria by Day 3. Wound closure rates, IVIS measurements, and survival data all demonstrate phage therapy efficacy and phage-antibiotic synergy.

TABLE 7
Mouse groups and treatment
(a) Mouse Groups

| | | | | | Treatments (0, |
|---|---|---|---|---|---|
| | | | | | 1, 2, 3, Days, IP |
| | | | | | and/or |
| Group # | # Mice | Wound? | Bacteria | Dose (CFU) | topically) |
| G1 | 8 | Yes | PAO1::lux | 1.0 × 10⁷ | Saline once a |
| | | | | | day (IP & |
| | | | | | topical): 4 |
| | | | | | treatments |
| G2 | 8 | Yes | PAO1::lux | 1.0 × 10⁷ | 410 mg/kg |
| | | | | | Ceftazidime |
| | | | | | (CAZ BID) (IP): 7 |
| | | | | | treatments |
| G3 | 8 | Yes | PAO1::lux | 1.0 × 10⁷ | PAM3T once a |
| | | | | | day (IP & |
| | | | | | topical): 4 |
| | | | | | treatments |
| G4 | 8 | Yes | PAO1::lux | 1.0 × 10⁷ | PAM3T once a |
| | | | | | day (Topical) & |
| | | | | | 410 mg/kg CAZ |
| | | | | | BID (IP): 4 & 7 |
| | | | | | treatments, |
| | | | | | respectively |

(b) Treatments

| | PBS | | CAZ | PAM3T | |
|---|---|---|---|---|---|
| Group | IP | Topical | IP | IP | Topical |
| G1 | Yes | Yes | No | No | No |
| G2 | No | No | 410 mg/kg | No | No |
| G3 | No | No | No | Yes | Yes |
| G4 | No | No | 410 mg/kg | No | Yes |

While 7/8 (87.5%) of control mice treated with saline died (not shown), the phage treatment protected 100% of mice from lethal septicemic infection (G3- and G4 group). PAM3T reduced the burden of P. aeruginosa PAO1::lux in infected wounds to undetectable levels within two days, and phage-treated wounds closed as rapidly as by Day 14, versus by Day 21 for positive control mice treated with ceftazidime.

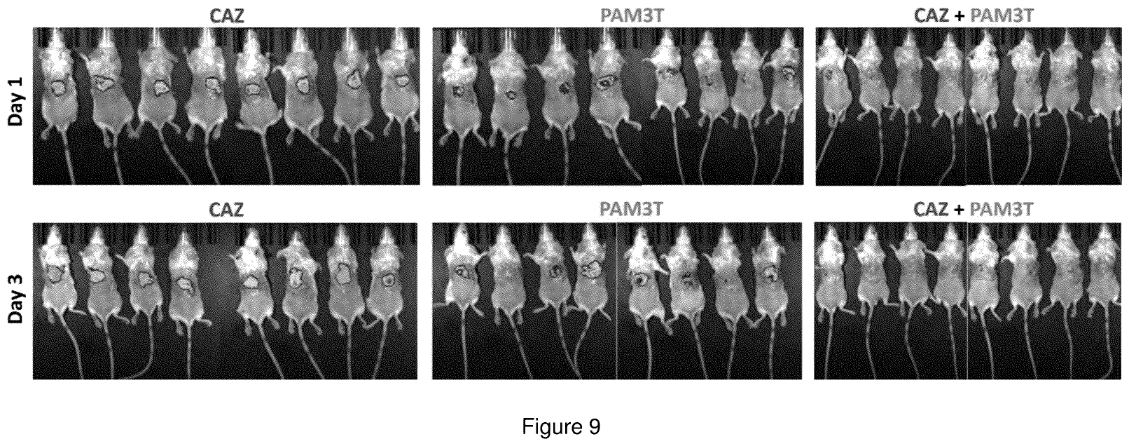

FIG. 9 shows IVIS images of PAO1::lux infection of dorsal wound 24 and 72 hours post treatment of wounds with antibiotic, phage cocktail, or both combined, (a) wound size and closure following treatment with saline, ceftazidime, PAM3T cocktail alone, or both ceftazidime and PAM3T and, (b) survival of mice infected with PAO1::lux following treatment with saline, ceftazidime, PAM3T, or a combination of ceftazidime and PAM3T.

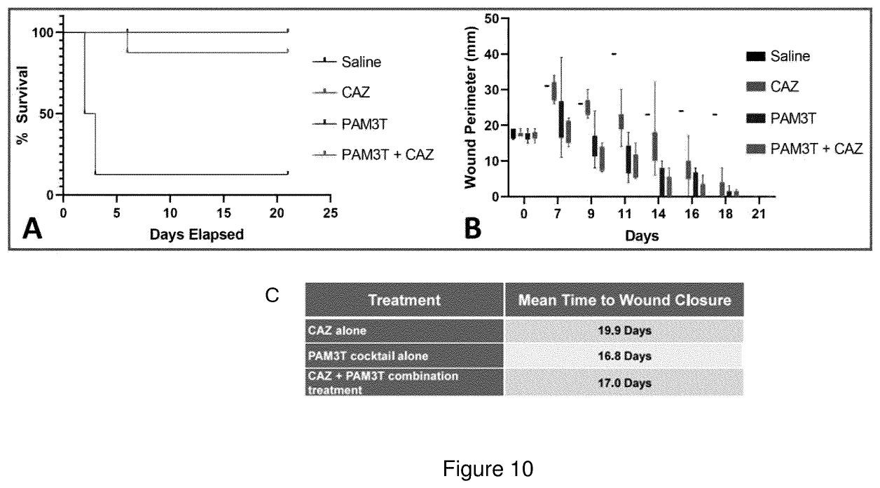

FIG. 10 presents FIG. 9 results in graphs, showing therapeutic effect of PAM3T treatment in a mouse wound infection model. FIG. 10A shows mouse survival rates. Only 1/8 (13%) of saline treated mice survived. As many as 7/8 (87%) of CAZ treated mice survived. All (8/8, 100%) PAM3T and PAM3T+CAZ combo treated mice survived. FIG. 10B is a graph showing wound healing (mean time to wound closure), and FIG. 10C table summarizes the results. i.e., it took 19.9 days in CAZ-treated mice, 16.8 days in PAM3T treated mice, and 17.0 days in PAM3T+CAZ co-treated mice, demonstrating the efficacy of PAM3T phage cocktail comprising the “trained phage”.

1. A method of expanding the activity of therapeutic phages, comprising steps of:
i. mixing taxonomically similar phages with complimentary lytic activity, wherein the taxonomically similar phages are, optionally, one or more selected from KEN1, KEN10, AFR43, KEN22, KEN25, KEN37, and KEN39;
ii. passaging mixed phages against a panel of phage-resistant bacterial clinical isolates, wherein the phage-resistant bacterial clinical isolates are, optionally, one or more selected from PAO1, MRSN 20176, MRSN414780, and MRSN 15882;
iii. pooling lysates from wells showing reduction in bacterial growth to form pooled lysate
iv. repeating steps (i)-(iii) using the pooled lysate;
v. identifying plaque formation on phage-resistant strains using pooled lysate;
vi. isolating phage clones from plaques formed in step (iv) to obtain candidate phages, and
vii. assessing host range of candidate phages; and
viii. optionally, assessing stability via serial propagation against single strain and re-assessing host range.

2. A method of developing phages having antibacterial activity in expanded range of multidrug resistant (MDR) strains of a bacterial species, wherein the bacterial species is Pseudomonas aeruginosa or Klebsiella pneumoniae, the method comprising steps of;
i. selecting at least two genetically similar phages, which have complementary lytic activity against a bacterial species, and determining the titer of each phage;
ii. mixing the phages at about the same ratio of each phage titer to make a phage cocktail to yield an input cocktail of 1×10⁵-1×10²⁰ pfu/mL, and optionally about 1×10¹⁰ pfu/mL;
iii. adding a volume of 10-20 MDR strains and a phage-susceptible strain of a bacterial species, which are individually cultured overnight in wells of a multi-well plate filled with a certain volume of bacterial culture media, in one strain per one column (or row) manner;
iv. performing a serial dilution of the phage cocktail of step ii (10⁰ to 10⁻⁷ or to 10⁻¹¹) in bacterial culture media;
v. adding each of the serial dilutions of step iv into a row (or a column) of the wells containing the bacterial culture of step iii;
vi. incubating the plate(s) with shaking, optionally at 37° C. overnight;
vii. pooling the lysate from the bacterial culture wells showing reduced bacterial growth or bacterial lysis;
viii. clearing the pooled lysates, optionally by vortex mixing with CHCl₃ and centrifugation;
ix. repeating steps iii to viii with the cleared lysate at least 5, 7, 10 or 15 rounds;
x. while performing step ix, periodically, optionally every 5 rounds, testing plaque forming activity with the cleared lysate, using a double-layered agar plate with a pan-phage-resistant strain;
xi. collecting individual phage plaques from the double-layered agar plate;
xii. performing steps iii to vii with each collected individual phages; and
xiii. selecting phages showing expanded host range when compared with parent phages;
wherein the method optionally further comprises
xiv. testing the selected phages against genetically diverse strain global diversity set;
xv. selecting phages from the phages of step xiv, which show expanded host range when compared with parent phages in step xiv;
xvi. isolating and purifying the phage particles, and sequencing their DNAs; and
xvii. testing phage stability with at least three clones of the selected phages; by re-testing them against the strain global diversity set to assess host range.

3. The method of claim 2, wherein at least two phages of Phikmvvirus genus are selected for the development of phages against MDR P. aeruginosa strains.

4. The method of claim 3, wherein the phages of Phikmvvirus genus are KEN1 (SEQ ID NO:28), KEN10 (SEQ ID NO: 29), and AFR43 (SEQ ID NO: 27).

5. The method of claim 2, wherein the phage susceptible P. aeruginosa strain is PAO1, or wherein the pan-phage resistant P. aeruginosa strain is MRSN 20176.

6. (canceled)

7. The method of claim 2, wherein at least two phages of Jiaodavirus genus are selected for the development of phages against MDR K. pneumoniae strains; wherein, optionally, the phages of Jiaodavirus genus are KEN22 (SEQ ID NO: 30), KEN25 (SEQ ID NO: 31), KEN37 (SEQ ID NO: 32), and KEN39 (SEQ ID NO: 33).

8. (canceled)

9. The method of claim 2, wherein the phage susceptible K. pneumoniae strain is MRSN414780 or wherein the pan-phage resistant K. pneumoniae strain is MRSN 15882.

10. (canceled)

11. A bacteriophage able to infect MDR strains of P. aeruginosa, wherein the phage comprises a nucleic acid sequence of any of SEQ ID NOs: 1-14, or a nucleic acid sequence of at least 97% sequence identity therewith and/or bacteriophage able to infect MDR strains of K. pneumoniae, wherein the phage comprises a nucleic acid sequence of any of SEQ ID NOs: 21-32 or a nucleic acid sequence of at least 97% sequence identity therewith.

12. (canceled)

13. A composition for the treatment of P. aeruginosa infection, wherein the composition comprises at least one phage selected from claim 11, and wherein, optionally, the composition comprises other phages against P. aeruginosa strains, said other phages being one, two, three, or four phages selected from genus of Pbunavirus, Nankokuvirus, and/or Abidjan virus.

14. (canceled)

15. The composition of claim 13, wherein the selected Pbunavirus is EPa11, EPa15, and/or Epa22.

16. The composition of claim 13, wherein the selected Nankoku virus is Epa16 and/or Epa18.

17. The composition of claim 13, wherein the selected Abidjan virus is Epa43.

18. The composition of claim 13, wherein the composition comprises EPa11, EPa15, Epa16, Epa18, and SEQ ID NO:3.

19. A composition for the treatment of K. pneumoniae infection, wherein the composition comprises at least one phage selected from claim 11.

20. (canceled)

21. (canceled)

22. (canceled)

23. (canceled)

24. (canceled)

25. (canceled)

26. (canceled)

27. (canceled)

28. (canceled)

29. (canceled)

30. A method of treating a subject having P. aeruginosa and/or K. pneumoniae infection, the method comprising administering an effective amount of a composition comprising at least one phage of claim 11, wherein administering comprises administration through a nasal, parenteral, or topical route.

31. (canceled)

32. The method of claim 30, wherein the subject has P. aeruginosa and/or K. pneumoniae infection in the lung, urinary tract, skin, and/or blood stream.

33. The method of claim 32, wherein the subject has P. aeruginosa and/or K. pneumoniae infection after lung transplant or is suffering from pneumonia, cystic fibrosis, bronchiectasis, bladder infection (cystitis), kidney infection (pyelonephritis), skin infection (cellulitis, burn wounds), and/or sepsis.

34. A method of disinfecting a surface, comprising applying to the surface a liquid, aerosol, or powder composition comprising any of the phages of claim 11.

35. A method of disinfecting a fish tank or aquafarm, comprising applying to the water the composition comprising any of the phages of claim 11, wherein the composition is formulated into a liquid, aerosol, or powder form.

36. A panel set of P. aeruginosa strains for phage training, comprising MRSN 317, 552, 1388, 2144, 6220, 6678, 8130, 8136, 8914, 12427, 13488, 16344, 20176, 20193, 25678, 26263, 358800, and PAO1; or a panel set of K. pneumoniae strains for phage training, comprising MRSN 4759, 6778, 15687, 15882, 22232, 27989, 479404, 511348, 614201, 681054, and 414780.
